(19) United States
(12) Patent Application Publication
(10) Pub. No.: US 2021/0244705 A1
(43) Pub. Date: Aug. 12, 2021

(54) THERAPEUTIC COMPOSITIONS, PRODUCTS OF MANUFACTURE AND METHODS FOR AMELIORATING OR PREVENTING CORONAVIRUS INFECTION

(71) Applicant: CENTRE FOR DIGESTIVE DISEASES, Five Dock (AU)

(72) Inventor: Thomas Julius BORODY, Five Dock (AU)

(21) Appl. No.: 17/116,942

(22) Filed: Dec. 9, 2020

Related U.S. Application Data
(63) Continuation-in-part of application No. 16/828,891, filed on Mar. 24, 2020.
(60) Provisional application No. 62/971,803, filed on Feb. 7, 2020.
(60) Provisional application No. 62/972,486, filed on Feb. 10, 2020.
(60) Provisional application No. 62/988,852, filed on Mar. 12, 2020.
(60) Provisional application No. 62/990,283, filed on Mar. 16, 2020.
(60) Provisional application No. 62/992,137, filed on Mar. 19, 2020.
(60) Provisional application No. 63/019,883, filed on May 4, 2020.
(60) Provisional application No. 63/060,461, filed on Aug. 3, 2020.
(60) Provisional application No. 63/109,214, filed on Nov. 3, 2020.

(51) Int. Cl. A61K 31/35; A61K 33/30; A61K 31/7052; A61K 31/47; A61K 31/375; A61K 31/593; A61K 9/00; A61K 9/20; A61K 9/14

(52) U.S. Cl. CPC A61K 31/35; A61K 33/30; A61K 31/7052; A61K 31/47; A61K 9/14; A61K 31/593; A61K 9/0053; A61K 9/0073; A61K 9/20; A61K 31/375

(57) ABSTRACT

In alternative embodiments, provided are pharmaceutical compositions comprising combinations of drugs, including products of manufacture and kits, and methods for using them, for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, combinations, or cocktails, of a drug or drugs as provided herein are administered either enterally, parenterally and/or by inhalation. In alternative embodiments, combinations, or cocktails, of drugs as provided herein are used to block intracellular metabolic pathways and prevent progression of the infection to clinical illness and death. In alternative embodiments, novel aerosol, spray or mist or powder formulations for inhalation are provided. In alternative embodiments, provided are therapeutic combinations of drugs or a drug, a pharmaceutical dosage form, a drug delivery device, or a product of manufacture, comprising: opaganib or YELIVA™, or opaganib or YELIVA™ and oral and/or inhaled chloroquine (or ARALEN™) chloroquine phosphate, chloroquine diphosphate and/or hydroxychloroquine (optionally, PLAQUENIL™), with or without azithromycin, wherein optionally each or all of the opaganib, the chloroquine (or ARALEN™), chloroquine phosphate, chloroquine diphosphate and/or hydroxychloroquine (optionally, PLAQUENIL™) and/or azithromycin, and others, are in or formulated as a formulation for inhalation, for example, formulated as an aerosol, spray, mist, liquid or powder.

### RELATED APPLICATIONS

This United States Utility Patent Application is a continuation-in-part application (“CIP”) of and claims priority under 35 U.S.C. § 120 to United States Patent Application Serial No. (USSN) 16/828,891, filed Mar. 24, 2020 (now pending), which claims benefit of priority under 35 U.S.C. § 119(e) to U.S. Ser. No. 62/971,803 filed Feb. 7, 2020; U.S. Ser. No. 62/972,486 filed Feb. 10, 2020; U.S. Ser. No. 62/988,852 filed Mar. 12, 2020; U.S. Ser. No. 62/990,283 filed Mar. 16, 2020; and U.S. Ser. No. 62/992,137 filed Mar. 19, 2020, and this application also claims priority under 35 U.S.C. § 119(e) to U.S. Ser. No. 63/019,883, May 4, 2020; U.S. Ser. No. 63/060,461 Aug. 3, 2020; and, U.S. Ser. No. 63/109,214, filed Nov. 3, 2020. The aforementioned applications are expressly incorporated herein by reference in their entirety and for all purposes.

### TECHNICAL FIELD

This invention generally relates to infectious diseases. In alternative embodiments, provided are pharmaceutical compositions comprising combinations of drugs, including products of manufacture and kits, and methods for using them, for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, combinations, or cocktails, of a drug or drugs as provided herein are administered either enterally, parenterally and/or by inhalation. In alternative embodiments, combinations, or cocktails, of drugs as provided herein are used to block intracellular metabolic pathways and prevent progression of the infection to clinical illness and death. In alternative embodiments, novel aerosol, spray or mist or powder formulations for inhalation are provided. In alternative embodiments, provided are therapeutic combinations of drugs or a drug, a pharmaceutical dosage form, a drug delivery device, or a product of manufacture, comprising: opaganib or YELIVA™, or opaganib or YELIVA™ and oral and/or inhaled chloroquine (or ARALEN™), chloroquine phosphate, chloroquine diphosphate and/or hydroxychloroquine (optionally, PLAQUENIL™), with or without azithromycin, wherein optionally each or all of the opaganib, the chloroquine (or ARALEN™), chloroquine phosphate, chloroquine diphosphate and/or hydroxychloroquine (optionally, PLAQUENIL™), and/or azithromycin, or other drugs, are in or formulated as a formulation for inhalation, for example, formulated as an aerosol, spray, mist, liquid or powder.

### BACKGROUND

Coronavirus infections have previously caused SARS (Severe Acute Respiratory Syndrome) and MERS (Middle East Respiratory Syndrome) and are particularly difficult to treat with anti-viral agents, and single drug regimens have not been found to be effective against the current coronavirus infection (2019-nCoV). The coronavirus infection (2019-nCoV), which started in China in December 2019, has spread rapidly throughout the world and has claimed hundreds of lives in China. The known coronavirus anti-infective agents used singly or alone are unable to cure the infection.

### SUMMARY

In alternative embodiments, provided are therapeutic combinations of drugs or a drug, a pharmaceutical dosage form, a drug delivery device, or a product of manufacture, comprising: ivermectin; an antibiotic, and zinc. In alternative embodiments of this therapeutic combination:
the antibiotic comprises a tetracycline class drug, and optionally the tetracycline class drug comprises doxycycline, and optionally the therapeutic combination comprises about 25 mg to about 600 mg doxycycline;
the antibiotic comprises azithromycin; and optionally the therapeutic combination comprises between about 50 mg to about 2000 mg azithromycin, or optionally the azithromycin comprise an oral extended-release formulation of azithromycin;
the zinc comprises a zinc sulphate, a zinc acetate, a zinc gluconate or a zinc picolinate, and optionally the therapeutic combination comprises between about 1 mg to 250 mg zinc;
the therapeutic combination further comprises a vitamin, optionally the vitamin comprises vitamin D or cholecalciferol, optionally dosaged at between about 3,000 to about 100,000 units vitamin D or cholecalciferol, and optionally the therapeutic combination comprises between about 10,000 to about 50,000 units vitamin D or cholecalciferol, and optionally the vitamin comprises vitamin C, and optionally the vitamin C is formulated or administered at a dosage of between about 500 to 5000 units (U) per dose;
the therapeutic combination is formulated as a liquid or an aerosol, or as a powder, or is formulated as a tablet, capsule, tablet or geltab, or is formulated as an injectable formulation, or an intramuscular (IM) or intravenous (IV) formulation;
the ivermectin is dosaged for administration at between about 3 to 240 mg per day; and/or
the therapeutic combination further comprises hydrocortisone, cortisol or dexamethasome, or further comprises hydroxychloroquine, for example, the therapeutic combination comprises ivermectin and hydroxychloroquine orchloroquine.

In alternative embodiments, provided are therapeutic combinations of drugs or a drug, a pharmaceutical dosage form, a drug delivery device, or a product of manufacture, comprising:

(a) opaganib or YELIVA™, or opaganib or YELIVA™ and oral and/or inhaled or aerosol chloroquine (or ARALEN™), chloroquine phosphate, chloroquine diphosphate and/or hydroxychloroquine (optionally, PLAQUENIL™), wherein optionally each or both of the opaganib and the chloroquine (or ARALEN™) chloroquine phosphate, chloroquine diphosphate and/or hydroxychloroquine (optionally, PLAQUENIL™) are in or formulated as a formulation for inhalation, for example, formulated as an aerosol, spray, mist, liquid or powder, or each or both are formulated for oral, intramuscular or intravenous administration, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage;

(b) lopinavir, ritonavir and oseltamivir (optionally, TAMIFLU™), and/or zanamivir (or RELENZA™);

(c) lopinavir combined (formulated) with ritonavir, or KALETRA™, ALTERA™, ALUVIA™, KALMELTREX, LOPIIVIUNE™ or LOPINAVIR™, and/or zanamivir (or RELENZA™), or lopinavir and ritonavir separately formulated;

(d) lopinavir combined (formulated) with ritonavir (or KALETRA™, ALTERA™, ALUVIA™, KALMELTREX, LOPIIVIUNE™ or LOPINAVIR™), or lopinavir and ritonavir, and oseltamivir (optionally, TAMIFLU™), and/or zanamivir (or RELENZA™), optionally also with inhaled or aerosol formulations or versions of chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously;

(e) lopinavir, ritonavir, chloroquine and oseltamivir (or TAMIFLU™); wherein optionally the chloroquine comprises inhaled or aerosol chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously;

(f) lopinavir and oseltamivir (optionally, TAMIFLU™), and/or zanamivir (or RELENZA™);

(g) ritonavir and oseltamivir (optionally, TAMIFLU™), and/or zanamivir (or RELENZA™);

(h) remdesivir (optionally, GS5734™, Gilead Sciences) alone, or oseltamivir (optionally, TAMIFLU™) and remdesivir (optionally, GS-5734™, Gilead Sciences), and optionally the remdesivir is an oral formulation and/or an inhaled or aerosol remdesivir formulation;

(i) oseltamivir (optionally, TAMIFLU™) and efavirenz (optionally, SUSTIVA™), and/or zanamivir (or RELENZA™);

(j) oseltamivir (optionally, TAMIFLU™) and nevirapine (or the combination efavirenz with emtricitabine and tenofovir, or ATRIPLA™);

(k) oseltamivir (or TAMIFLU™) and amprenavir (optionally, AGENERASE™);

(l) oseltamivir (optionally, TAMIFLU™) and nelfinavir (optionally, VIRACEPT™); or

(m) a thiazolide class drug, optionally nitazoxanide (optionally ALINIA™, NIZONIDE™) or tizoxanide (or 2-Hydroxy-N-(5-nitro-2-thiazolyl)benzamide), with or in combination with any of (a) to (hh), or any drug or drug combination as provided herein, optionally a thiazolide class drug, optionally nitazoxanide, with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; or a thiazolide class drug (optionally, nitazoxanide or tizoxanide) and oseltamivir (or TAMIFLU™), and optionally the thiazolide class drug (optionally, nitazoxanide or tizoxanide) is formulated or administered with ribavirin or tribavirin (or COPEGUS™, REBETOL™, or VIRAZOLE™), and an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;
(n) plitidepsin (also known as dehydrodidemnin B), or APLIDIN™ (PharmaMar, S.A.);

(o) an inhibitor or S-phase kinase-associated protein 2 (SKP2), or dioscin, or niclosamide, or NICLOCIDE™, FENASAL™, or PHENASAL™;

(p) ribavirin or tribavirin (or COPEGUS™, REBETOL™, or VIRAZOLE™) interferon beta 1b, or a combination of ribavirin and interferon beta, or a combination of lopinavir and ritonavir and interferon-beta-1b;

(q) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz (optionally, SUSTIVA™), elvitegravir, emtricitabine, enfuvirtide, entecavir, epirubicin, epoprostenol, etravirine, famciclovir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II and/or interferon type III), lamivudine, lopinavir, loviride, ledipasvir, leronlimab, maraviroc, methisazone, moroxydine, nelfinavir, nevirapine, nexavir, nitazoxanide (optionally ALINIA™, NIZONIDE™), norvir, a nucleoside analogue (optionally brincidofovir, didanosine, favipiravir (also known as T-705, avigan, or favilavir, Toyama Chemical, Fujifilm, Japan), vidarabine, galidesivir (optionally, BCX4430, IMMUCILLIN-A™), remdesivir (optionally, GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™) or any combination thereof;

(r) an mucolytic therapy or drug, optionally acetylcysteine, ambroxol, bromhexine, carbocisteine, erdosteine, mecysteine or dornase alfa, or an expectorant, optionally guaifenesin;

(s) a viral, or a coronavirus or a COVID-19, protease inhibitor, optionally ASCO9 (CAS registry no. 1000287-05-7) (Janssen Research and Development, LLC), ritonavir or ASCO9 and ritonavir, or a JAK1/2 inhibitor (optionally baricitinib), optionally compound 11r (University of Lubeck, Germany, see optionally, Zhang et al J. Med Chem 2020, Feb. 11, 2020), or darunavir, cobicistat or darunavir and cobicistat;

(t) an angiotensin-converting enzyme 2 (ACE2) inhibitor, optionally to block the site of viral spike protein interaction for anti-SARS-CoV-2 infection control;

(u) an anti-vascular endothelial growth factor (VEGF) (optionally VEGF-A) drug or antibody, optionally bevacizumab;

(v) a protease inhibitor, optionally danoprevir, optionally a serine protease inhibitor, optionally camostat or narlaprevir (optionally ARLANSA™);

(w) anti-PD-1 checkpoint inhibitor, optionally camrelizumab;

(x) a compound or antibody capable of binding complement factor C5 and blocking membrane attack complex formation, optionally eculizumab;

(y) a cathepsin inhibitor, optionally a cathepsin K, B or L inhibitor, optionally relacatib;

(z) thalidomide, or thalidomide and glucocorticoid (optionally low-dose glucocorticoid), or and thalidomide and celecoxib;

(aa) an antibacterial antibiotic or a macrolide drug, wherein optionally the macrolide drug comprises azithromycin, optionally dosaged at between about 50 mg to about 2000 mg per dose or per day (optionally, ZITHROMAX™, or AZITHROCIN™, optionally an oral extended- or delayed-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,

and optionally the antibacterial antibiotic comprises a tetracycline class drug, a glycylcycline or a fluorocycline class drug, or an analogue thereof, and optionally the tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline;

doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof,

and optionally the antibacterial antibiotic or macrolide drug, optionally azithromycin, is administered in combination with, and/or is combined with, chloroquine (or ARALEN™), chloroquine phosphate, chloroquine diphosphate and/or hydroxychloroquine (optionally, PLAQUENIL™), and the combination is administered commencing on the first, second, third, fourth, fifth, sixth, seventh, eighth, ninth and/or tenth day of therapy, or is administered for 1, 2, 3, 4, 5, 6, 7, 8, 9, 10 or up to 20 or more days, or for between about 1 to 21 days or longer, or is administered until within 1, 2, 3, 4, 5, 6, 7, 8, 9, 10 or up to 20 or more days of ending the therapy for treating, preventing, ameliorating, slowing the progress of, decreasing the severity of or preventing the coronavirus infection,

and optionally the chloroquine (or ARALEN™), chloroquine phosphate, chloroquine diphosphate and/or hydroxychloroquine (optionally, PLAQUENIL™) is administered the entire length of the treatment but the azithromycin, optionally dosaged at between about 50 mg to about 2000 mg per dose or per day (optionally, ZITHROMAX™, or AZITHROCIN™, optionally an oral extended-release formulation of azithromycin, or ZMAX™) administration is halted or ceased after two, three, four, five or six days after treatment is commenced, and optionally the azithromycin administration is replaced by a tetracycline class drug, and optionally the tetracycline class drug comprises doxycycline, or DORYX™, DOXYHEXA™ DOXYLIN™ administration,

and optionally the antibacterial antibiotic, optionally azithromycin (optionally, ZITHROMAX™, or AZITHROCIN™, optionally dosaged at between about 50 mg to about 2000 mg per dose or per day, and optionally an oral extended-release formulation of azithromycin, or ZMAX™), is administered or formulated with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or cholecalciferol (vitamin D3) or calcifediol,

and optionally the antibacterial antibiotic comprises an antimycobacterial drug, and optionally the antimycobacterial drug comprises clofazimine (optionally LAMPRENE™);

(bb) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 240 mg per day,

optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder,

and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is formulated with and/or administered with chloroquine (or ARALEN™), chloroquine phosphate, chloroquine diphosphate and/or hydroxychloroquine (optionally, PLAQUENIL™) with or without zinc (optionally a zinc sulphate, acetate, gluconate or picolinate), and optionally this combination is administered weekly, or every two week, or one every 5 to 28 days, as a prophylactic,

and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,

and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered alone for 1, 2, 3, 4, 5, 6, 7, 8, 9, 10 or up to 20 or more days, followed by administration of an antibiotic (optionally doxycycline) for a corresponding period of days, and optionally repeating the cycle of dosaging,

and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is formulated or administered with:
(i) at least one antibiotic (wherein optionally the antibiotic is doxycycline (optionally, DORYX™, DOXYHEXA™, DOXYLIN™) (optionally formulated or administered at a dosage of between about 25 mg to 600 mg per dose or per day), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
(ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
(iii) a zinc (optionally a zinc sulphate, acetate, gluconate or picolinate) optionally formulated or administered at a dosage of between about 1 mg to 250 mg; and/or
(iv) at least one vitamin, and optionally the at least one vitamin comprises: vitamin C optionally formulated or administered at a dosage of between about 500 to 5000 units (U) per dose, and/or Vitamin D (or cholecalciferol) optionally formulated or administered at a dosage of between about 3,000 to 100,000 units per day, or between about 10,000 to 50,000 units a day,
and optionally the avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered or formulated alone or in combination with any of the above (i) to (iv) (for example, at least one antibiotic, chloroquine (or ARALEN™) chloroquine phosphate, chloroquine diphosphate and/or hydroxychloroquine (optionally, PLAQUENIL™), zinc and/or at least one vitamin are formulated (and administered) as oral formulations (for example, as tablets, capsules, gels or geltabs), injectable formulations, powders (for example, for inhalation or for addition to an ingestible liquid) or liquids (for example, for ingestion, infusion or injection);

(cc) chloroquine (or ARALEN™), chloroquine phosphate, chloroquine diphosphate and/or hydroxychloroquine (optionally, PLAQUENIL™) alone or with (or formulated with) or in combination with any of (a) to (bb), or chloroquine, chloroquine phosphate, chloroquine diphosphate and/or hydroxychloroquine (optionally, PLAQUENIL™) and oseltamivir (or TAMIFLU™);

(dd) chloroquine (optionally, ARALEN™), chloroquine phosphate, chloroquine diphosphate and/or hydroxychloroquine (optionally, PLAQUENIL™) alone or with:
(i) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 3 to 340 mg per day, or about 6 mg to 60 mg, or about 10 mg to 80 mg dosages, or about 12 to 50 mg dosages;
(ii) vitamin D, vitamin D2 (or ergocalciferol), vitamin D3 (or cholecalciferol) optionally at a dosage of between about 3,000 to 100,000 units per day, or between about 10,000 to 50,000 units a day, and/or
(iii) with (i) and (ii) and zinc (optionally a zinc sulphate, acetate, gluconate or picolinate) optionally at a dosage of between about 1 mg to 250 mg, or (iv) the combination of (iii) also with a tetracycline class drug, wherein optionally the tetracycline class drug comprises doxycycline, or DORYX™ DOXYHEXA™, DOXYLIN™, optionally dosages at between about 25 mg to 600 mg per day or per dose, optionally between about 100 mg to 500 mg, or a between about 200 mg to 400 mg per dose or per day;

(ee) colchicine, or COLCRYS™, MITIGARE™;

(ff) a corticosteroid class drug such as budesonide (optionally RHINOCORT™ or PULMICORT™), prednisolone (or ORAPRED™), methyl-prednisolone, prednisone (or DELTASONE™ or ORASONE™) or hydrocortisone (or CORTEF™),

and optionally the corticosteroid class drug (for example budesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,

and optionally the corticosteroid class drug is formulated as a powder or for administration in an inhaler or by nasal spray, or for rectal administration,

and optionally the corticosteroid class drug (for example, budesonide) is administered together with or in combination with 10 mg to 80 mg, an antibiotic (optionally azithromycin or a tetracycline class drug,

wherein optionally the tetracycline class drug comprises doxycycline, or DORYX™, DOXYHEXA™, DOXYLIN™), zinc and/or a vitamin (optionally vitamin D or calcifediol, D2 (or ergocalciferol), D3 (or cholecalciferol), C, E, B12, B6);

(gg) an anti-androgen drug, optionally bicalutamide, optionally CASODEX™ and optionally the anti-androgen,

and optionally bicalutamide is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;

(hh) a hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);

(ii) an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2),

and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin or a tetracycline class drug, wherein optionally the tetracycline class drug comprises doxycycline, or DORYX™, DOXYHEXA™, DOXYLIN™); chloroquine (or ARALEN™), chloroquine phosphate, chloroquine diphosphate and/or hydroxychloroquine (optionally, PLAQUENIL™); zinc; remdesivir (optionally, GS-5734™, Gilead Sciences); oseltamivir (or TAMIFLU™); and/or, hydrocortisone; or, any combination thereof;

(jj) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™, DOXYHEXA™, DOXYLIN™;

(kk) a dendrimer, optionally astodrimer sodium (Starpharma, Melbourne, Australia);

(ll) an antihistamine class drug such as azelastine, or ASTELIN™, OPTIVAR™, ALLERGODIL™, brompheniramine, fexofenadine or ALLEGRA™ pheniramine or AVIL™, or chlorpheniramine;

(mm) a selective serotonin reuptake inhibitor (SSRI) class drug, optionally fluvoxamine, or LUVOX™, FAVERIN™, FLUVOXIN™; and/or

(nn) any combination of (a) to (mm),

and optionally any one or several or all of (a) to (nn) with an (or formulated with or formulated as an) inhaled or aerosol formulation such as a powder or a mist or aerosol, and/or formulated with or formulated as an oral, intramuscular (IM) or intravenous (IV) formulation, wherein optionally both the inhaled (or aerosol) and the oral, IV and/or IM formulations are administered simultaneously or sequentially,

and optionally the inhaled or aerosol formulation comprises chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) administered simultaneously or overlapping,

and optionally the inhaled or aerosol formulation comprises an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,

and optionally any one or several or all of (a) to (nn), or any therapeutic combination of drugs or a drug, or a pharmaceutical dosage form as provided herein, are administered orally, intramuscularly, subcutaneously, topically, by use of an enema, intravaginally, or intravenously, or administration is by subcutaneous administration, sublingual administration, inhalation or by aerosol (optionally by inhalation of a liquid, an aerosol, a spray, a mist or a powder), by absorbable patch, by use of an implant, or by use of an enema or a suppository.

In alternative embodiments of the therapeutic combinations of the drugs or drug, pharmaceutical dosage form, drug delivery device, or product of manufacture as provided herein:
the drugs or drug, pharmaceutical dosage form, drug delivery device, or product of manufacture further comprise (optionally, are formulated with, or are administered with) an (or an additional) anti-viral drug or medication, or anti-microbial drug, or palliative agent or drug,

wherein optionally the anti-viral drug or medication, or anti-microbial drug, is or comprises: molnupiravir, efavirenz (optionally, SUSTIVA™), tenofovir, emtricitabine and tenofovir, nevirapine (or the combination efavirenz with emtricitabine and tenofovir, or ATRIPLA™), amprenavir (optionally, AGENERASE™), nelfinavir (optionally, VIRACEPT™) and/or remdesivir (optionally, GS-5734™, Gilead Sciences), a viral RNA-dependent RNA polymerase inhibitor, optionally galidesivir, and optionally the anti-viral drug or medication is or comprises an anti-retroviral drug or drug combination, and optionally the anti-retroviral drug or drug combination comprises: darunavir and cobicistat (optionally, REZOLSTA™ or PREZCOBIX™); atazanavir and cobicistat (or EVOTAZ™); abacavir, lamivudine and dolutegravir (TRIUMEQ™); tenofovir (or disoproxil or emtricitabine) and elvitegravir and cobicistat (optionally, STRIBILD™); tenofovir (or disoproxil or emtricitabine) and elvitegravir and cobicistat (COMPLERA™ or EVIPLERA™); molnupiravir, efavirenz (optionally, SUSTIVA™), emtricitabine and tenofovir (ATRIPLA); lamivudine, nevirapine and stavudine (optionally, TRIOMUNE™); atazanavir and cobicistat (optionally, EVOTAZ™); lamivudine and raltegravir (optionally, DUTREBIS™); lamivudine and dolutegravir (or DOVATO™); doravirine, lamivudine and tenofovir (optionally, DELSTRIGO™); or lamivudine, zidovudine and nevirapine (optionally, CUOVIR-N™);

and optionally the additional anti-viral drug or medication, or anti-microbial drug, is formulated with the chloroquine (optionally, ARALEN™), chloroquine phosphate, chloroquine diphosphate, hydroxychloroquine (optionally, PLAQUENIL™), lopinavir, ritonavir and/or oseltamivir or is formulated separately from the chloroquine (optionally, ARALEN™), chloroquine phosphate, chloroquine diphosphate, hydroxychloroquine (optionally, PLAQUENIL™), lopinavir, ritonavir and/or oseltamivir,

and optionally the anti-viral drug or medication, or anti-microbial drug, or palliative agent comprises or further comprises: magnesium (Mg, optionally administer intravenously (IV) to maintain a blood concentration of between about 2.0 and 2.4 mmol/1); zinc (optionally a zinc sulphate, acetate, gluconate or picolinate, optionally administered at about 75 to 100 mg/day or at a dosage of between about 1 mg to 250 mg); at least one vitamin, wherein optionally the at least one vitamin comprises vitamin K, vitamin D or calcifediol (optionally D2 (or ergocalciferol) or Vitamin D3 or cholecalciferol), optionally administered at about 1000 to 4000 ugm/day), vitamin B6 (or pyridoxine), vitamin B12, vitamin E, and/or vitamin C (optionally administered at 500 mg bid); a flavonoid, plant flavonol or quercetin optionally administered at between about 250 to 500 mg bid; atorvastatin, or LIPITOR™, SORTIS™ (optionally administered at between about 40 mg/day to 80 mg/day); or, melatonin, or CIRCADIN™, SLENYTO™ (optionally between about 6 to 12 mg a day, optionally, at night), any of which are optionally given enterally or parenterally;
the chloroquine (optionally, ARALEN™), chloroquine phosphate, chloroquine diphosphate, hydroxychloroquine (optionally, PLAQUENIL™) lopinavir, ritonavir and/or oseltamivir are formulated separately or together, or the lopinavir and ritonavir are formulated together and the oseltamivir is formulated separately;
the therapeutic combination of drugs or drug, pharmaceutical dosage form, or the chloroquine (optionally, ARALEN™), chloroquine phosphate, chloroquine diphosphate, hydroxychloroquine (optionally, PLAQUENIL™), lopinavir, ritonavir and/or oseltamivir and/or the anti-viral drug or medication, or anti-microbial drug are formulated or contained in a liquid formulation (optionally sterile saline or water), a spray, a powder, an aerosol, mist, or any formulation for inhalation, a pill, a capsule, a tablet, or a geltab, or equivalents;
and optionally the therapeutic combination of drugs or drug, pharmaceutical dosage form, or the chloroquine (optionally, ARALEN™), chloroquine phosphate, chloroquine diphosphate, hydroxychloroquine (optionally, PLAQUENIL™) lopinavir, ritonavir and/or oseltamivir and/or the anti-viral drug or medication, or anti-microbial drug, are coated on the surface of or contained in: a bead, a powder, a particle, or a multilayered bead or particle, and optionally the bead, powder, particle or the multilayered bead or particle is contained in a pill, a capsule, a tablet, or a geltab, or equivalents, for oral delivery, wherein optionally the pill, capsule, tablet, geltab or equivalent for oral delivery is a hard gelatin capsule or equivalent, or comprises a hard gelatin or equivalent; and/or
the lopinavir, ritonavir and oseltamivir are formulated or packaged for administration as or dosing in the ratio of 25:100:75 of lopinavir: ritonavir: oseltamivir.

In alternative embodiments, provided are drug delivery devices or packages, a kit, a blister package, a clamshell or a tray, comprising a therapeutic combination of drugs or drug, a pharmaceutical dosage form or a formulation as provided herein, wherein the drug delivery device comprises an inhalation device, nebulizer, puffer device, or inhaler or a nasal spray device, and optionally the inhaler, nebulizer, puffer device, or nasal spray device is a hand-held device, for example, a hand-held inhaler or nasal spray device, and optionally the hand-held device (optionally, inhaler, nebulizer or nasal spray device) is a metered or dose-counting inhaler or a nasal spray device,

wherein the drug delivery device or package, blister pack, clamshell or tray comprises a plurality of compartments spatially arranged on the drug delivery device or package, blister pack, clamshell or tray to follow a dosage administration regimen, wherein the spatially arranged plurality of compartments are in at least two rows, each row marked for the time for which a drug of the drug combination, or the tablets, pills, capsules, geltabs or equivalents, are to be taken by a user (optionally a patient), optionally one row marked for morning, breakfast or AM administration, and one row marked for evening, dinnertime or PM administration, and optionally the row or rows marked for morning, breakfast or AM administration is or are positioned above the row or rows marked for evening, dinnertime or PM administration,

and optionally the spatially arranged plurality of compartments are in four rows, two rows marked for morning, breakfast or AM administration, and two rows marked for evening, dinnertime or PM administration,

and optionally the spatially arranged plurality of compartments are arranged to facilitate and/or direct the patient to take the drugs in that row two times a day (bid), three times a day (tid), four times a day, or up to ten times a day (wherein optionally the higher amounts are for the very sick), optionally also comprising indication of actual times for patient to ingest the drugs in each row, and if appropriate, directions to also ingest fluids or other drugs and/or food (for example, small amounts of food),

and optionally each row comprises seven compartments for one dosage administration for each day of the week, or eight compartments for one dosage administration for each day of the week and one spare, and optionally each vertically arranged set of compartments, or columns, are marked for which day of the week the dosage formulations contained therein are to be taken by the user, and optionally where the drug delivery device or package, blister pack, clamshell or tray has one row for morning, breakfast or AM administration, and one row marked for evening, dinnertime or PM administration, each column or day will have two compartments, optionally where the compartment for morning, breakfast or AM administration is above the compartment for evening, dinnertime or PM administration,

and optionally each compartment has a foil or equivalent backing, or each compartment is an environmentally- (optionally moisture-, pathogen-, and/or light-) protected or sealed storage unit, and optionally the foil backing requires minimal finger strength to remove a dosage formulation (optionally one, two or three or more capsules, tablets, pills, geltabs or equivalents) in the compartment,

and optionally the blister package is a face seal blister package, a gang run blister package, a mock blister package, an interactive blister package or a slide blister package,

and optionally the drug delivery device or package, blister package, clamshell or tray is joined with board material which allows the product to be packaged, handled, hung, displayed and/or shipped without damaging the blister protection or seal, and optionally also provided with child resistant features,

and optionally the drug delivery device or package, blister package, clamshell or tray comprises a medical electronic monitory system that records administration time and transmits information to near-field communication (NFC) enabled mobile phone,

wherein optionally the kit is a travel kit comprising instructions of use by the traveler, wherein the instructions optionally comprise instructing the traveler to immediately take an indicated dosage, optionally a first arrange row of drugs on the drug delivery device or package, blister package, clamshell or tray, if they believe they have been in contact with or in near contact with or exposed to an individual that is infect or might be infected with coronavirus (for example, a COVID-19) or to an individual having fever, sore throat, rigors or shakes, chest pain on breathing, coughing, diarrhea and/or muscle aches.

In alternative embodiments, provided are methods for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the sub-family Orthocoronavirinae, or a virus in the family Coronaviridae, or a virus in the order Nidovirales, comprising administered to an individual in need thereof a therapeutic combination of drugs or drug, a pharmaceutical dosage form, a drug delivery device, or a product of manufacture as provided herein, wherein optionally the therapeutic combinations of drugs or drugs or pharmaceutical dosage forms are administered as inhaled or aerosol formulations (for example, powders, liquids, aerosols sprays, mists) and/or are administered (optionally simultaneously, or sequentially) with oral, intravenous (IV) or intramuscular formulations,

and optionally for preventive or prophylactic purposes the therapeutic combination of drugs or drug, or the pharmaceutical dosage form, or the drug delivery device or the product of manufacture is used or administered: daily, every other day, every third day, every fourth day, every fifth day, every sixth day, or once a week, or monthly.

In alternative embodiments of the methods, the administered therapeutic combination of drugs or drug, pharmaceutical dosage form, drug delivery device, or product of manufacture comprises, or the method of administration comprises:

(a) opaganib or YELIVA™, or opaganib or YELIVA™ and oral and/or inhaled or aerosol chloroquine (or ARALEN™), chloroquine phosphate, chloroquine diphosphate and/or hydroxychloroquine (optionally, PLAQUENIL™) wherein optionally each or both of the opaganib and the chloroquine (or ARALEN™) chloroquine phosphate, chloroquine diphosphate and/or hydroxychloroquine (optionally, PLAQUENIL™) are in or formulated as a formulation for inhalation, for example, formulated as an aerosol, spray, mist, liquid or powder, or each or both are formulated for oral, intramuscular or intravenous administration, and optionally each are simultaneously administered in both oral and in inhalation forms, or only is administered as an inhalant;

(b) lopinavir, ritonavir and oseltamivir (optionally, TAMIFLU™), and/or zanamivir (or RELENZA™);

(c) lopinavir combined (formulated) with ritonavir, or KALETRA™, ALTERA™, ALUVIA™, KALMELTREX, LOPIIVIUNE™ or LOPINAVIR™, or lopinavir and ritonavir separately formulated;

(d) lopinavir combined (formulated) with ritonavir, or KALETRA™, ALTERA™, ALUVIA™, KALMELTREX, LOPIIVIUNE™ or LOPINAVIR™, and oseltamivir (optionally, TAMIFLU™), optionally also with inhaled or aerosol chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously,

wherein optionally the dosage administration comprises: lopinavir about 200 mg twice daily, ritonavir about 50 mg twice daily, chloroquine about 250 mg twice daily, oseltamivir (TAMIFLU™) about 75 mg twice daily;

(e) lopinavir, ritonavir and oseltamivir (or TAMIFLU™) (and/or zanamivir (or RELENZA™)); with inhaled or aerosol chloroquine (or ARALEN™), chloroquine phosphate, chloroquine diphosphate and/or hydroxychloroquine (optionally, PLAQUENIL™) and/or oral chloroquine (or ARALEN™), chloroquine phosphate, chloroquine diphosphate and/or hydroxychloroquine (optionally, PLAQUENIL™) simultaneously;

(f) lopinavir and oseltamivir (optionally, TAMIFLU™), and/or zanamivir (or RELENZA™);

(g) ritonavir and oseltamivir (optionally, TAMIFLU™), and/or zanamivir (or RELENZA™);

(h) remdesivir (optionally, GS5734™, Gilead Sciences) alone, or oseltamivir (optionally, TAMIFLU™) and remdesivir (optionally, GS-5734™, Gilead Sciences);

(i) oseltamivir (optionally, TAMIFLU™) and efavirenz (optionally, SUSTIVA™), molnupiravir and/or zanamivir (or RELENZA™);

(j) oseltamivir (optionally, TAMIFLU™) and nevirapine (or the combination efavirenz or molnupiravir with emtricitabine and tenofovir, or ATRIPLA™);

(k) oseltamivir (or TAMIFLU™) and amprenavir (optionally, AGENERASE™);

(l) oseltamivir (optionally, TAMIFLU™) and nelfinavir (optionally, VIRACEPT™); or

(m) a thiazolide class drug, optionally nitazoxanide (or ALINIA™ NIZONIDE™) or tizoxanide (or 2-Hydroxy-N-(5-nitro-2-thiazolyl)benzamide), with or in combination with any of (a) to (hh), or any drug or drug combination as provided herein, optionally a thiazolide class drug, optionally nitazoxanide, with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and optionally the thiazolide class drug (optionally, nitazoxanide or tizoxanide) and oseltamivir (or TAMIFLU™), and optionally the thiazolide class drug (optionally, nitazoxanide or tizoxanide) is formulated or administered with ribavirin or tribavirin (or COPEGUS™, REBETOL™, or VIRAZOLE™), and an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin,

and optionally formulated and administered as an inhalant or a mist (optionally using a nebulizer, nasal spray or equivalent);

(n) plitidepsin (also known as dehydrodidemnin B), or APLIDIN™ (PharmaMar, S.A.);

(o) an inhibitor or S-phase kinase-associated protein 2 (SKP2), or dioscin, or niclosamide, or NICLOCIDE™, FENASAL™, or PHENASAL™;

(p) ribavirin or tribavirin (or COPEGUS™, REBETOL™, or VIRAZOLE™) interferon beta 1b, or interferon alfa-2b, or a combination of ribavirin and an interferon, for example, interferon alpha or beta (optionally, interferon beta 1b or interferon alfa-2b), or a combination of lopinavir and ritonavir and an interferon, e.g., interferon-beta-1b and/or interferon alfa-2b;

(q) abacavir, acyclovir optionally, (ACICLOVIR™), adefovir, amantadine, ampligen, amprenavir (optionally, AGENERASE™), aprepitant, arbidol, atazanavir, atripla, balavir, baloxavir marboxil (XOFLUZA™), bepotastine, bevirimat, bictegravir, biktarvy, brilacidin, cidofovir, caspofungin, lamivudine and zidovudine (optionally, COMBVIR™), cobicstat, colisitin, cocaine, darunavir, delavirdine, descovy, didanosine, docosanol, dolutegravir, ecoliever, edoxudine, efavirenz (optionally, SUSTIVA™), elvitegravir, emtricitabine, enfuvirtide, entecavir, epirubicin, epoprostenol, etravirine, famciclovir, favilavir, fomivirsen, fosamprenavi, foscarnet, fosfonet, galidesivir, ibacitabine, icatibant, idoxuridine, ifenprodil, imiquimod, imunovir, indinavir, inosine, an interferon (optionally interferon type I, interferon type II, interferon alfa-2b, and/or interferon type III), lamivudine, lopinavir, loviride, ledipasvir, leronlimab, maraviroc, methisazone, molnupiravir, moroxydine, nelfinavir, nevirapine, nexavir, nitazoxanide (optionally ALINIA™, NIZONIDE™) norvir, a nucleoside analogue (optionally didanosine, vidarabine, galidesivir (optionally, BCX4430, IMMUCILLIN-A™), remdesivir (optionally, GS-5734™, Gilead Sciences), cytarabine, gemcitabine, emtricitabine, lamivudine, zalcitabine, abacavir, acyclovir, entecavir, stavudine, telbivudine, zidovudine, idoxuridine and/or trifluridine or any combination thereof), oseltamivir (or TAMIFLU™), peginterferon alfa-2a or alfa-2b, penciclovir, peramivir (optionally, RAPIVAB™), perfenazine, pleconaril, plurifloxacin, podophyllotoxin, pyramidine, raltegravir, rifampicin, ribavirin or tribavirin (or COPEGUS™, REBETOL™, or VIRAZOLE™), rilpivirine, rimantadine, ritonavir, saquinavir, sofosbuvir, stavudine, telaprevir, tegobuv, tenofovir alafenamide, tenofovir disoproxil, tenofovir, tipranavir, trifluridine, trizivir, tromantadine, truvada, valaciclovir (optionally, VALTREX™), valganciclovir, valrubicin, vapreotide, vicriviroc, vidarabine, viramidine, velpatasvir, vivecon, zalcitabine, zanamivir (optionally, RELENZA™), zidovudine, an immunosuppressive drug (optionally tocilizumab or atlizumab, or ACTEMRA™, or ROACTEMRA™), zanamivir (or RELENZA™), or any combination thereof;

(r) chloroquine (or ARALEN™), chloroquine phosphate, chloroquine diphosphate and/or hydroxychloroquine (optionally, PLAQUENIL™) with or in combination with any of (a) to (p), or chloroquine chloroquine phosphate, chloroquine diphosphate and/or hydroxychloroquine (optionally, PLAQUENIL™) and oseltamivir (or TAMIFLU™);

(s) an mucolytic therapy or drug, optionally acetylcysteine, ambroxol, bromhexine, carbocisteine, erdosteine, mecysteine or dornase alfa, or an expectorant, optionally guaifenesin;

(t) a viral, or a coronavirus or a COVID-19, protease inhibitor, optionally ASCO9 (CAS registry no. 1000287-05-7) (Janssen Research and Development, LLC), ritonavir or ASCO9 and ritonavir, or a JAK1/2 inhibitor (optionally baricitinib), optionally compound 11r (University of Lubeck, Germany, see for example, Zhang et al J. Med Chem 2020, Feb. 11, 2020), or darunavir, cobicistat or darunavir and cobicistat;

(u) an angiotensin-converting enzyme 2 (ACE2) inhibitor, optionally to block the site of viral spike protein interaction for anti-SARS-CoV-2 infection control;

(v) an anti-vascular endothelial growth factor (VEGF) (optionally VEGF-A) drug or antibody, optionally bevacizumab;

(w) a protease inhibitor, optionally danoprevir, optionally a serine protease inhibitor, optionally camostat or narlaprevir (optionally ARLANSA™);

(x) anti-PD-1 checkpoint inhibitor, optionally camrelizumab;

(y) a compound or antibody capable of binding complement factor C5 and blocking membrane attack complex formation, optionally eculizumab;

(z) a cathepsin inhibitor, optionally a cathepsin K, B or L inhibitor, optionally relacatib;

(aa) thalidomide, or thalidomide and glucocorticoid (optionally low-dose glucocorticoid), or and thalidomide and celecoxib;

(bb) an antibacterial antibiotic or a macrolide drug,

wherein optionally the macrolide drug comprises azithromycin (optionally, ZITHROMAX™, or AZITHROCIN™, optionally dosaged at between about 50 mg to about 2000 mg per dose or per day, optionally an oral extended-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,

and optionally the macrocyclic lactone antibiotic comprises an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally formulated and administered as an inhalant or a mist (optionally using a nebulizer, nasal spray or equivalent)

and optionally the macrolide drug, optionally azithromycin, is administered at a dosage of about 50 to 200 mg a day, or at about 25 to 100 mg three times a day (tid), or at 50 mg tid,

wherein optionally the antibacterial antibiotic or the macrolide drug. optionally azithromycin, is formulated for normal release or as a delayed release formulation,

and optionally the azithromycin (optionally, ZITHROMAX™, or AZITHROCIN™, optionally dosaged at between about 50 mg to about 2000 mg per dose or per day, optionally an oral extended-release formulation of azithromycin, or ZMAX™) is administered for only the first, second, third, fourth, fifth, sixth or seventh day of treatment, and optionally azithromycin administration is ceased and replaced with an antiviral antibiotic, optionally a tetracycline class drug, wherein optionally the tetracycline class drug comprises doxycycline, or DORYX™ DOXYHEXA™, DOXYLIN™,

and optionally the antibacterial antibiotic comprises a tetracycline class drug, a glycylcycline or a fluorocycline class drug, or an analogue thereof, and optionally the tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof,

and optionally the antibacterial, optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™), is administered with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and cholecalciferol (vitamin D3) or calcifediol,

and optionally the antibacterial antibiotic comprises an antimycobacterial drug, and optionally the antimycobacterial drug comprises clofazimine (optionally LAMPRENE™);

(cc) an avermectin class drug such as ivermectin (optionally STROMECTOL™, SOOLANTRA™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally dosaged and/or administered at about 5 microgram/kg to about 1 gram (g) per day, optionally formulated or administered at about 1 to 10, 12, 15, 20, 30, 40, 50, 60, 70, 80, 100, 120, 140, 160, 180, 200, 220 or 240 mg per day, or between about 1 to 240 mg per day, or between about 3 to 240 mg per day,

optionally formulated or administered with an antibiotic (optionally azithromycin, minocycline, amoxicillin, niclosamide, nitazoxanide, hydroxychloroquine or doxycycline, and optionally the doxycycline is at between about 25 to 600 mg per dose or per day, or at about 100 mg per dose or per day, and optionally the azithromycin is at between about 50 mg to 2000 mg per dose or per day), optionally as a single or a divided dose, and optionally formulated and administered as an inhalant or a mist (optionally using a nebulizer, nasal spray or equivalent), optionally formulated as an aerosol, spray, mist, liquid or powder, optionally formulated as an aerosol, spray, mist, liquid or powder,

and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated with and/or administered with chloroquine (or ARALEN™), chloroquine phosphate, chloroquine diphosphate and/or hydroxychloroquine (optionally, PLAQUENIL™) with or without zinc, and optionally this combination is administered weekly, or every two week, or one every 5 to 28 days, as a prophylactic,

and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin is administered alone in the morning (AM), and an antibiotic (optionally doxycycline) and/or a chloroquine (optionally, ARALEN™), chloroquine phosphate, chloroquine diphosphate and/or hydroxychloroquine (optionally, PLAQUENIL™) is administered in the afternoon and/or evening,

and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered alone for 1, 2, 3, 4, 5, 6, 7, 8, 9, 10 or up to 20 or more days, followed by administration of an antibiotic (optionally doxycycline) for a corresponding period of days, and optionally repeating the cycle of dosaging,

and optionally an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is formulated or administered, optionally at an about 5 mg to 200 mg per dose, or between about 1 to 240 mg per day or mg per dose, alone or in combination with one, several or all of:
(i) at least one antibiotic (wherein optionally the antibiotic is doxycycline (optionally, DORYX™, DOXYHEXA™, DOXYLIN™) (optionally formulated or administered at a dosage of between about 25 mg to 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg);
(ii) chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);
(iii) a zinc (optionally a zinc sulphate, acetate, gluconate or picolinate) optionally formulated or administered at a dosage of between about 1 mg to 250 mg; and/or
(iv) at least one vitamin, and optionally the at least one vitamin comprises: vitamin C optionally formulated or administered at a dosage of between about 500 to 5000 units (U) per dose, and/or Vitamin D (or cholecalciferol or calcifediol) optionally formulated or administered at a dosage of between about 3,000 to 100,000 units per day, or between about 10,000 to 50,000 units a day,
and optionally the an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, is administered or formulated alone or in combination with any of the above (i) to (iv) (for example, at least one antibiotic, chloroquine (or ARALEN™) chloroquine phosphate, chloroquine diphosphate and/or hydroxychloroquine (optionally, PLAQUENIL™), zinc and/or at least one vitamin are formulated (and administered) as oral formulations (for example, as tablets, capsules, gels or geltabs), injectable formulations, powders (for example, for inhalation or for addition to an ingestible liquid) or liquids (for example, for ingestion, infusion or injection),
and optionally the an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, alone or in combination with any of the above (i) to (iv) (for example, at least one antibiotic, chloroquine (or ARALEN™), chloroquine phosphate, chloroquine diphosphate and/or hydroxychloroquine (optionally, PLAQUENIL™), zinc and/or at least one vitamin are administered once a month (or every 2, 3, 4, 5, or 6 weeks), optionally as a preventive measure (or for prophylactic purposes), which can be administered to individuals who have or who have not been vaccinated,
and optionally a combination of: (1) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, (2) at least one antibiotic (wherein optionally the antibiotic is doxycycline (optionally, DORYX™, DOXYHEXA™ DOXYLIN™) (optionally formulated or administered at a dosage of between about 25 mg to about 600 mg, or between about 100 mg to about 500 mg), or azithromycin (optionally, ZITHROMAX™, or AZITHROCIN™, optionally dosaged at between about 50 mg to about 2000 mg per dose or per day, optionally an oral extended-release formulation of azithromycin, or ZMAX™) (optionally formulated or administered at a dosage of between an about 50 mg to 2000 mg), (3) a zinc (optionally a zinc sulphate, acetate, gluconate or picolinate) optionally formulated or administered at a dosage of between about 1 mg to 250 mg; and (4) at least one vitamin, and optionally the at least one vitamin comprises: vitamin C optionally formulated or administered at a dosage of between about 500 to 5000 units (U) per dose, and/or Vitamin D (or cholecalciferol or calcifediol) optionally formulated or administered at a dosage of between about 3,000 to 100,000 units per day, or between about 10,000 to 50,000 units a day, are administered in an overlapping dosage pattern (optionally overlapping by 1, 2, 3, 4, 5, 6, 7, 8, 9, or 10 days) or non-overlapping dosaging, overlapping or followed by administration of: (2), (3) and (4) (for example, any avermectin such as ivermectin, doxycycline and/or azithromycin, zinc, vitamins C and D) but where (1) is replaced by: chloroquine (or ARALEN™), chloroquine phosphate, chloroquine diphosphate and/or hydroxychloroquine (optionally, PLAQUENIL™) (optionally formulated or administered at a dosage of between an about 10 mg to 2000 mg per day);

(dd) chloroquine (optionally, ARALEN™), chloroquine phosphate, chloroquine diphosphate and/or hydroxychloroquine (optionally, PLAQUENIL™) alone or with: (i) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 10 mg to 80 mg dosages, or 12 to 60 mg dosages, and/or (ii) vitamin D, vitamin D2 (or ergocalciferol), vitamin D3 (or cholecalciferol or calcifediol) optionally at a dosage of between about 3,000 to 100,000 units per day (or about 10,000 to 50,000 units per day, or (iii) with (i) and (ii) and zinc (optionally a zinc sulphate, acetate, gluconate or picolinate) optionally at a dosage of between about 1 mg to 250 mg, or (iv) the combination of (iii) also with a tetracycline class drug, wherein optionally the tetracycline class drug comprises doxycycline, or DORYX™, DOXYHEXA™ DOXYLIN™, optionally dosages at between about 25 mg to 600 mg per day, optionally between about 100 mg to about 500 mg or between about 200 mg to about 400 mg;

(ee) any one or several or all of drugs of (a) to (dd), or any therapeutic combination of drugs or drug, pharmaceutical dosage form of any of the preceding claims, are administered as an inhaled aerosol, spray, mist, powder or liquid or other inhalation formulation, or are administered with inhaled (for example, aerosol or power administered by inhaler) chloroquine (or ARALEN™), chloroquine phosphate, chloroquine diphosphate and/or hydroxychloroquine (optionally, PLAQUENIL™)

and optionally the chloroquine, chloroquine phosphate, chloroquine diphosphate and/or hydroxychloroquine administration is started (early, first) 1, 2, 3, 4, 5, 6, 7, 8, 9, 10 or up to 20 or more days before administration of the one or several of the drugs of any of (a) to (dd), wherein optionally this initial (early, first) administration of chloroquine, chloroquine phosphate, chloroquine diphosphate and/or hydroxychloroquine administration is by inhalation and/or by an oral, IM or IV formulation, or administration is by subcutaneous administration, sublingual administration, inhalation (optionally by inhalation of a liquid, an aerosol, a spray, a mist or a powder), by absorbable patch, by use of an implant, or by use of an enema or a suppository,

and optionally the chloroquine, chloroquine phosphate, chloroquine diphosphate and/or hydroxychloroquine administration is started with a high dose (for example, a so-called “loading dose”), for example, an oral (for example, a single dose such as a single tablet, capsule or pill), IV or IM dosage of between about 250 mg, 300 mg, 350 mg, 300 mg or 0.5 gram (gm) (500 mg) and 1.5 gm, or between about 400 mg and 1 gm, optionally with follow up administrations every 4 to 10 days, or every 1, 2, 3, 4, 5, 6, 7, 8, 9, 10 days or up to 20 or more days, at a lower dosage of between about 50 gm to 200 mg, or about 100 mg, total daily dosage, optionally continuing for between about one week to one month,

wherein optionally the initial (early, first) administration of the chloroquine, chloroquine phosphate, chloroquine diphosphate and/or hydroxychloroquine also comprises early, first administration of the macrolide drug, optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™), and optionally the macrolide drug is started with a high dose (for example, a so-called “loading dose”), optionally an oral (for example, a single dose such as a single tablet, capsule or pill), IV or IM dosage of between about 400 mg to 0.5 gram (gm) (500 mg) and 1 gm, or at about 500 mg, optionally with follow up administrations every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month,

wherein optionally the initial (early, first) administration of the chloroquine, chloroquine phosphate, chloroquine diphosphate and/or hydroxychloroquine and/or the macrolide drug also comprises early, first administration of opaganib or YELIVA™, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage,

and optionally are administered with oral (for example, pills, tablets, capsules, liquids) chloroquine (or ARALEN™), chloroquine phosphate, chloroquine diphosphate and/or hydroxychloroquine (optionally, PLAQUENIL™)

wherein the inhaled or aerosol (for example, aerosol spray, mist, or power administered by inhaler) therapeutic combination of drugs or drug, pharmaceutical dosage form, drug delivery device, or product of manufacture is or are administered simultaneously or overlapping or sequentially; or

(ff) colchicine, or COLCRYS™, MITIGARE™;

(gg) a corticosteroid class drug such as budesonide (optionally RHINOCORT™ or PULMICORT™), prednisolone (or ORAPRED™), methyl-prednisolone, prednisone (or DELTASONE™ or ORASONE™) or hydrocortisone (or CORTEF™),

and optionally the corticosteroid class drug (for example budesonide) is administered by inhalation, for example, in a nebulized form, for example, between about 1 mg to 12 mg per day of budesonide is administered by inhalation, or between about 6 to 80 mg per day of prednisolone is administered orally, or between about 6 to 100 mg per day of prednisone is administered orally, or between about 30 to 400 mg per day of hydrocortisone is administered orally,

and optionally the corticosteroid class drug is formulated as a powder or for administration in an inhaler or by nasal spray, or for rectal administration,

and optionally the corticosteroid class drug (for example, budesonide) is administered together with or in combination with 10 mg to 80 mg, an antibiotic (optionally azithromycin or a tetracycline class drug

and optionally the corticosteroid class drug (for example budesonide) is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, an antibiotic (optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™) or a tetracycline class drug, optionally doxycycline), zinc and/or a vitamin (optionally vitamin D, D2 (or ergocalciferol), D3 (or cholecalciferol or calcifediol), C, E, B12, B6);

(hh) an anti-androgen drug, optionally bicalutamide, optionally CASODEX™, and optionally the anti-androgen, optionally bicalutamide is administered together with or in combination with an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin;

(ii) a hydrocortisone or cortisol (optionally CORTEF™, SOLUCORTEF™), optionally hydrocortisone sodium succinate or hydrocortisone acetate or dexamethasome (optionally DEXTENZA™, OZURDEX™, NEOFORDEX™);

(jj) an alpha-ketoamide (α-ketoamide), wherein optionally the alpha-ketoamide is a structure as described by Zhang et al, J. Med. Chem. 2020, 63, 9, 4562-4578, or Meng et al Chem. Sci. (2019) vol. 10, pg 5156 (optionally the structure KAM-2), and optionally the alpha-ketoamide is formulated or administered as an inhalant or a powder or mist, and optionally formulated or administered with (optionally as an inhalant): an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; an antibiotic (optionally azithromycin (optionally, ZITHROMAX™, or AZITHROCIN™, optionally dosaged at between about 50 mg to about 2000 mg per dose or per day, optionally an oral extended-release formulation of azithromycin, or ZMAX™) or a tetracycline class drug, wherein optionally the tetracycline class drug comprises doxycycline); chloroquine (or ARALEN™), chloroquine phosphate, chloroquine diphosphate and/or hydroxychloroquine (optionally, PLAQUENIL™); zinc; remdesivir (optionally, GS-5734™, Gilead Sciences); oseltamivir (or TAMIFLU™); and/or, hydrocortisone; or, any combination thereof;

(kk) a compound, drug or formulation that decreases stomach acid production or decreases stomach pH, wherein optionally the compound, drug or formulation comprises famotidine, or PEPCID™, and optionally the famotidine is administered at a dosage of between about 10 to 60 mg per day, or between about 20 to 40 mg per day, and optionally the famotidine is administered is administered with: an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, and/or a tetracycline tetracycline class drug, and optionally the tetracycline class drug comprises doxycycline, or DORYX™, DOXYHEXA™, DOXYLIN™;

wherein optionally any combination of (a) to (jj) simultaneously administered in both inhaled (for example, aerosol spray, mist, or power administered by inhaler or by aerosol) and oral (for example, pills, tablets, capsules, liquids), intramuscular, subcutaneous (SC) and/or intravenous (IV) forms or formulations;

(ll) a dendrimer, optionally astodrimer sodium (Starpharma, Melbourne, Australia);

(mm) an antihistamine class drug such as azelastine, or ASTELIN™, OPTIVAR™, ALLERGODIL™, brompheniramine, fexofenadine or ALLEGRA™ pheniramine or AVIL™, or chlorpheniramine;

(nn) a selective serotonin reuptake inhibitor (SSRI) class drug, optionally fluvoxamine, or LUVOX™, FAVERIN™, FLUVOXIN™; and/or

(oo) an antibody or antisera capable of binding to an neutralizing a coronavirus, optionally COVID-19, wherein optionally the antibody or antisera is derived from an individual recovered from a coronavirus infection, and optionally the antibody is a recombinant antibody, optionally bamlanivimab (Lilly); and/or

(pp) any combination of (a) to (oo),

and/or the any one or several or all of (a) to (pp), or any therapeutic combination of drugs or a drug, or a pharmaceutical dosage form as provided herein, are administered orally, intramuscularly, subcutaneously, topically, by enema, intravaginally, or intravenously, or administration is by subcutaneous administration, sublingual administration, inhalation or by aerosol (optionally by inhalation of a liquid, an aerosol, a spray, a mist or a powder), by absorbable patch, by use of an implant, or by an enema or a suppository,

and optionally the inhaled or aerosol formulation comprises an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin.

In alternative embodiments of the methods:

the lopinavir, ritonavir and oseltamivir are formulated or packaged for administration as or dosing in the ratio of 25:100:75 of lopinavir: ritonavir: oseltamivir;

the chloroquine (optionally, ARALEN™), chloroquine phosphate, chloroquine diphosphate, hydroxychloroquine (optionally, PLAQUENIL™) lopinavir, ritonavir and/or oseltamivir, and/or the anti-viral drug or medication, or anti-microbial drug, are administered enterally or parenterally, or are administered orally, intramuscularly (IM), intravenously (IV), by inhalation, or by inhalation when formulated as an aerosol, a mist, a spray, a microparticle, a nanoparticle, or a powder;

the chloroquine (optionally, ARALEN™), chloroquine phosphate, chloroquine diphosphate, hydroxychloroquine (optionally, PLAQUENIL™) lopinavir, ritonavir and/or oseltamivir, and/or the anti-viral drug or medication, or anti-microbial drug, are administered one to ten times per day (for example, bid, tid, or 5, 6, 7, 8, 9, or 10 or more times a day) depending on the stage of the condition (for example, exposed to infection, positivity in blood but asymptomatic, or symptomatic, mild or severe;

the method comprises first administering (alone) for about 7 days (or 2, 3, 4, 5, 6 or 7 or more days) oseltamivir (or TAMIFLU™) at 3 times per day (tid) (or alternatively twice a day (bid) or four times a day or more), followed by a blood sample to be taken for virus testing; and if and when virus blood positivity is confirmed, or viral infection is otherwise confirmed, the osteltamivir is supplemented by the lopinavir and ritonavir (which can be administered together or separately) three times daily (tid) (or alternatively twice a day (bid) or four times a day or more), while continuing the osteltamivir, for example, all three medications three (or two or four or more) times daily or 9 (or more) medications daily;
the duration of the combined drug therapy, or the chloroquine (optionally, ARALEN™), chloroquine phosphate, chloroquine diphosphate or hydroxychloroquine (optionally, PLAQUENIL™) therapy, is about 2, 3, 4, 5, 6, 7, 8, 9, 10, 11, 12, 13, 14, 15, 16, 17, 18, 19, 20 or 21 or more or more days or for as long as the patient tests positive for the coronavirus (for example, a COVID-19) infection, or for at least 2, 3, 4, 5, 6, 7, 8, 9, 10, 11, 12, 13, 14, 15, 16, 17, 18, 19, 20 or 21 or more or more days after the patient no longer tests positive for coronavirus (for example, a COVID-19), or wherein the combined drug therapy, or

the chloroquine (optionally, ARALEN™), chloroquine phosphate, chloroquine diphosphate or hydroxychloroquine (optionally, PLAQUENIL™) therapy, begins (commences) as soon as an individual (a patient) learns (understands) he or she was in close proximity to a coronavirus (for example, a COVID-19) positive individual, or believes that he or she may have been in close proximity to a coronavirus (for example, a COVID-19) positive individual, or begins (commences) as soon as the individual (the patient) test positive for coronavirus (for example, a COVID-19), or

the chloroquine (optionally, ARALEN™), chloroquine phosphate, chloroquine diphosphate or hydroxychloroquine (optionally, PLAQUENIL™) therapy, continues until at least one, or at least two, tests show the individual (the patient) is negative for coronavirus (for example, a COVID-19), or continues until all body fluids or samples (optionally respiratory or oral swabs or fluids, sputum, serology or blood or serum, and stool or fecal samples) test negative for coronavirus (for example, a COVID-19), and optionally the individual (the patient) is retested at least every week, biweek or month to test or reinfection or reemergence or reoccurrence of viral infection or activity, and if the individual (the patient) again tests positive, then reinitiate treatment as used when the individual was first diagnosed with coronavirus (for example, a COVID-19) infection;

the combined drug therapy, or the chloroquine (optionally, ARALEN™) chloroquine phosphate, chloroquine diphosphate or hydroxychloroquine (optionally, PLAQUENIL™) therapy, comprises administering the chloroquine (optionally, ARALEN™), chloroquine phosphate, chloroquine diphosphate or hydroxychloroquine (optionally, PLAQUENIL™):
(a) until, or increasing the dosage of the chloroquine (optionally, ARALEN™), chloroquine phosphate, chloroquine diphosphate or hydroxychloroquine (optionally, PLAQUENIL™), until a steady state chloroquine (optionally, ARALEN™), chloroquine phosphate, chloroquine diphosphate or hydroxychloroquine (optionally, PLAQUENIL™) plasma concentration of at least 1 μg/mL (or at least approximately 3.125 μM/L) is achieved, or until a whole blood concentration of at least about 16 μM/L is achieved, and optionally sustained, until the patient's viremia becomes undetectable; or
(b) intravenously (IV) at a dosage of between about 10 mg/kg to 20 mg/kg, or at about 15 mg/kg, over a one to 6 hour period, or over four hour period, and optionally the IV infusion is repeated, optionally every 6 to 12 hours, optionally for between about 2 to 20 repeat infusions;

the combined drug therapy, or the chloroquine (optionally, ARALEN™) chloroquine phosphate, chloroquine diphosphate or hydroxychloroquine (optionally, PLAQUENIL™) therapy, is formulated with or is administered before, during or after: an anti-coronavirus vaccine or a vaccination with an anti-coronavirus (e.g., an anti-COVID-19) vaccine, or administration of a passive immunity therapy (for example, infusion (for example, intravenous infusion, for example, infusion of a hyperimmune globulin) of one or more antibodies capable of specifically binding to or neutralizing or stopping the multiplication or spread of a coronavirus (for example, a COVID-19), for example bamlanivimab (Lilly), which comprises man-made antibodies that are similar to the antibodies of patients who recovered from COVID-19;

the combined drug therapy, or the chloroquine (optionally, ARALEN™) chloroquine phosphate, chloroquine diphosphate or hydroxychloroquine (optionally, PLAQUENIL™) therapy, is administered with (optionally before, during and/or after), or formulated with, an anti-inflammatory therapy or at least one anti-inflammatory therapy drug, wherein optionally the anti-inflammatory therapy or drug comprises: a sphingosine kinase-2 (SK2) selective inhibitor (optionally, opaganib (optionally, YELIVA™), sirolimus, a JAK1/2/TYK2 inhibitor (optionally ruxolitinib), an anti-CD47 mAb (optionally meplazumab), COX (optionally, COX2) inhibitor, a glucocorticoid and/or an immunosuppressive agent, and optionally the immunosuppressive agent comprises tocilizumab or fingolimod; and/or

the combined drug therapy, or the chloroquine (optionally, ARALEN™) chloroquine phosphate, chloroquine diphosphate or hydroxychloroquine (optionally, PLAQUENIL™) therapy, is formulated with or is administered with (optionally before, during and/or after) administration of:
(a) an mucolytic therapy or drug, optionally acetylcysteine, ambroxol, bromhexine, carbocisteine, erdosteine, mecysteine or dornase alfa, or an expectorant, optionally guaifenesin;
(b) a viral, or a coronavirus or a COVID-19, protease inhibitor, optionally ASCO9 (CAS registry no. 1000287-05-7) (Janssen Research and Development, LLC), ritonavir or ASCO9 and ritonavir, or a JAK1/2 inhibitor (optionally baricitinib), optionally compound 11r (University of Lubeck, Germany, see for example, Zhang et al J. Med Chem 2020, Feb. 11, 2020), or darunavir, cobicistat or darunavir and cobicistat;
(c) an angiotensin-converting enzyme 2 (ACE2) inhibitor, optionally to block the site of viral spike protein interaction for anti-SARS-CoV-2 infection control;
(d) an anti-vascular endothelial growth factor (VEGF) (optionally VEGF-A) drug or antibody, optionally bevacizumab;
(e) a protease inhibitor, optionally danoprevir, optionally a serine protease inhibitor, optionally camostat or narlaprevir (optionally ARLANSA™);
(f) anti-PD-1 checkpoint inhibitor, optionally camrelizumab;
(g) a compound or antibody capable of binding complement factor C5 and blocking membrane attack complex formation, optionally eculizumab;
(h) a cathepsin inhibitor, optionally a cathepsin K, B or L inhibitor, optionally relacatib;
(i) thalidomide, or thalidomide and glucocorticoid (optionally low-dose glucocorticoid), or and thalidomide and celecoxib,
(j) an antibacterial antibiotic or a macrolide drug,

wherein optionally the macrolide drug comprises azithromycin (optionally, ZITHROMAX™, or AZITHROCIN™, optionally dosaged at between about 50 mg to about 2000 mg per dose or per day, optionally an oral extended-release formulation of azithromycin, or ZMAX™), clarithromycin (optionally, BIAXIN™), erythromycin (optionally, ERYTHROCIN™), or fidaxomicin (optionally, DIFICID™ or DIFICLIR™), troleandomycin (optionally, TEKMISIN™), tylosin (optionally, TYLOCINE™ or TYLAN™), solithromycin (optionally, SOLITHERA™), oleandomycin (or SIGMAMYCINE™), midecamycin, roxithromycin, kitasamycin or turimycin, josamycin, carbomycin or magnamycin, and/or spiramycin,

and optionally the azithromycin (optionally, ZITHROMAX™, or AZITHROCIN™, optionally an oral extended-release formulation of azithromycin, or ZMAX™) is administered for only the first, second, third, fourth, fifth, sixth or seventh day of treatment, and optionally azithromycin administration is ceased and replaced with an antiviral antibiotic, optionally a tetracycline class drug, wherein optionally the tetracycline class drug comprises doxycycline, or DORYX™ DOXYHEXA™, DOXYLIN™,

wherein optionally the antibacterial antibiotic or the macrolide drug. optionally azithromycin, is formulated for normal release or as a delayed release formulation,

and optionally the antibacterial antibiotic comprises a tetracycline class drug, a glycylcycline or a fluorocycline class drug, or an analogue thereof, and optionally the tetracycline, glycylcycline or fluorocycline drug or analogue thereof comprises or is: tetracycline or SUMYCIN™; chlortetracycline or AUREOMYCIN™; oxytetracycline; demeclocycline or DECLOMYCIN™, DECLOSTATIN™, LEDERMYCIN™, BIOTERCICLIN™, DEGANOL™, DETECLO™, DETRAVIS™, MECICLIN™, MEXOCINE™, CLORTETRIN™; lymecycline; meclocycline; metacycline; minocycline or MINOCIN™; rolitetracycline; doxycycline, or DORYX™, DOXYHEXA™, DOXYLIN™; tigecycline or TYGACIL™; eravacycline or XERAVA™; sarecycline or SEYSARA™; omadacycline or NUZYRA™; or any combination thereof;

(k) an allogenic stem cell or an allogenic stem cell therapy, wherein the allogenic stem cell therapy comprises use of an allogeneic mesenchymal stem cell, or remestemcel-L or RYONCIL™, and optionally the allogenic stem cell is administered by intravenous administration or infusion or

(l) any combination of (a) to (k).

In alternative embodiments, provided are uses of a therapeutic combination of drugs or drug, a pharmaceutical dosage form, a drug delivery device, or a product of manufacture as provided herein for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, provided are therapeutic combinations of drugs or a drug, a pharmaceutical dosage form, a drug delivery device, or a product of manufacture as provided herein for use in treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, provided are uses of a therapeutic combination of drugs or a drug, a pharmaceutical dosage form, a drug delivery device, or a product of manufacture as provided herein for the manufacture of a medicament, wherein optionally the medicament is used for treating, preventing, ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

The details of one or more exemplary embodiments of the invention are set forth in the description below. Other features, objects, and advantages of the invention will be apparent from the description, figure and from the claims.

All publications, patents, patent applications cited herein are hereby expressly incorporated by reference in their entireties for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 illustrates an exemplary product of manufacture of the invention, an exemplary blister pack. As illustrated the “oseltamivir”, or yellow highlighted, section of the blister pack is intended to be taken by the a patient who may have been in contact with an individual infected with Coronavirus, such as an individual having a history of meeting or coming into contact with an infected person, or who may have been in contact with an individual already having a fever and/or a symptom of a respiratory illness such as cough or shortness of breath, but the patient as yet has not received the results of a blood test (or any diagnostic test) for Coronavirus. The patient given the exemplary blister pack, and while waiting for his or her test result immediately begins taking a high dose 5 times daily of oseltamivir (which should have few adverse effects on the patient), which should begin slowing the spread and/or replication of the virus in the patient (assuming the patient is in fact infected). As soon as the patient if confirmed by a blood test (or any diagnostic test) to be infected with Coronavirus, or after five days on treatment with oseltamivir, the patient immediately moves on to the next part (section) of the blister pack, which in alternative embodiment has three or more drugs, for example: lopinavir, ritonavir and oseltamivir; lopinavir combined (formulated) with ritonavir, or lopinavir and ritonavir separately formulated; lopinavir combined (formulated) with ritonavir, and oseltamivir; lopinavir and oseltamivir; ritonavir and oseltamivir; oseltamivir and remdesivir, and the like as provided herein. A doctor can decide the frequency of dosing based on the clinical presentation of the patient and test results. If the blood (or other) test is negative for coronavirus, the patient may return the blister pack or keep it (or keep taking the oseltamivir, until a second 2^{nd} test (which can be the same or a different test) is also negative.

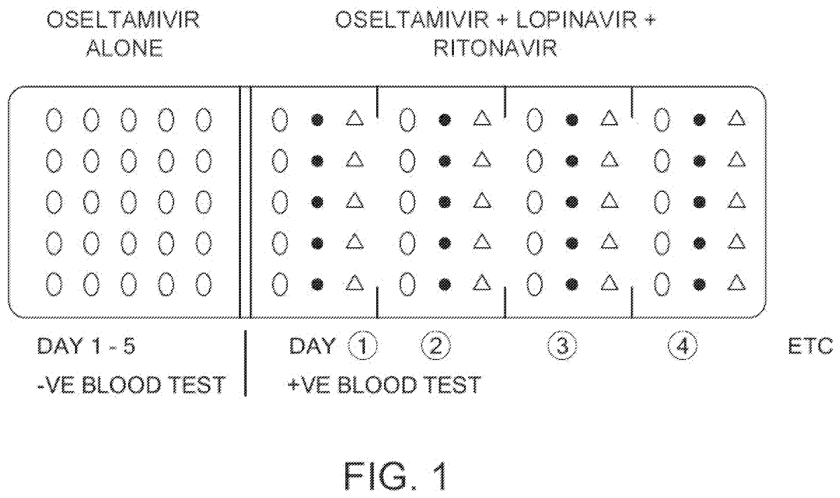

The drawings set forth herein are illustrative of exemplary embodiments provided herein and are not meant to limit the scope of the invention as encompassed by the claims.

Like reference symbols in the various drawings indicate like elements.

### DETAILED DESCRIPTION

In alternative embodiments, provided are pharmaceutical compositions comprising combinations of drugs, including products of manufacture and kits, and methods for using them, for treating, preventing (as a prophylaxis), ameliorating, slowing the progress of, decreasing the severity of or preventing a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales. In alternative embodiments, combinations, or cocktails, of drugs as provided herein are used to block intracellular metabolic pathways and intracellular viral replication, and prevent progression of the infection to clinical illness and death.

In alternative embodiments, provided are combinations of different medications which are used together can treat, ameliorate, slow the progress of, decrease the severity of or prevent the current (2019-nCoV) infections. In alternative embodiments, provided are novel methods of administration dosing to cover the period of exposure, diagnosis, and treatment.

In alternative embodiments, provided is combination of drugs comprising lopinavir combined (formulated) with ritonavir, or KALETRA™, ALTERA™, ALUVIA™, KALMELTREX, LOPIIVIUINE™ or LOPINAVIR™, and oseltamivir (or TAMIFLU™). In alternative embodiments, provided is combination of drugs comprising lopinavir, ritonavir and oseltamivir (or TAMIFLU™), and/or zanamivir (or RELENZA™)

In alternative embodiments, provided are products of manufacture such as blister packs or equivalents (for example, a clamshell, a tray, a shrink wrap and the like) comprising lopinavir combined (formulated) with ritonavir and oseltamivir, or lopinavir, ritonavir and oseltamivir, and/or zanamivir (or RELENZA™). In alternative embodiments, the products of manufacture, for example, blister pack, a clamshell, a tray, a shrink wrap and the like, arranges the drugs such that all drugs are taken together, or the oseltamivir is taken before the lopinavir combined (formulated) with ritonavir, or lopinavir and ritonavir.

In alternative embodiments, provided are methods for using combination of drugs and products of manufacture as provided herein comprising first administering to an individual (for example, an individual suspected of being exposed to the coronavirus, for example, an individual having a history of meeting or coming into contact with an infected person, or an individual already having a fever and/or a symptom of a respiratory illness such as cough or shortness of breath), oseltamivir (or TAMIFLU™) immediately and tests sent off for the coronavirus, with addition of (at least) lopinavir combined (formulated) with ritonavir, or lopinavir and ritonavir (separate formulations) to the administered drug regime when the test is positive for coronavirus (which can be within one, two, three or four or more days); and optionally also administering this regimen if the individual continues to have symptoms and is clinically judged to have coronavirus albeit (even if there is a) negative test.

Alternatively these drugs can be administered in the reverse order, i.e., first administer lopinavir combined (formulated) with ritonavir, or lopinavir and ritonavir (separate formulations), followed by (within one, two, three or four days, optionally if the individual continues to have symptoms) addition of administration of at least a third agent oseltamivir (or TAMIFLU™).

In alternative embodiments, dosing can be in the ratio of 25:100:75 of lopinavir: ritonavir: oseltamivir. For example, 25 mg (lopinavir), 100 mg (ritonavir), 75 mg (oseltamivir) respectively, or in multiples thereof, are administered one to ten times per day (for example, bid, tid, or 5, 6, 7, 8, 9, or 10 or more times a day) depending on the stage of the condition (for example, exposed to infection, positivity in blood but asymptomatic, or symptomatic, mild or severe. In alternative embodiments, oseltamivir, lopinavir and ritonavir are administered in increased amounts, for example, if the individuals condition does not improve, or does not improve quickly.

In alternative embodiments, products of manufacture as provided herein further comprise, or methods as provided herein further comprise use (administration of): molnupiravir, efavirenz (optionally, SUSTIVA™), nevirapine (or the combination efavirenz with emtricitabine and tenofovir, or ATRIPLA™), amprenavir (optionally, AGENERASE™), nelfinavir (optionally, VIRACEPT™) and/or remdesivir (optionally, GS5734™, Gilead Sciences). In alternative embodiments, one, several or all of these are concomitantly used in medications, for example, one, several or all of these can also be used in (formulated with) a drug composition or formulation or product of manufacture as provided herein, or can be used or administered separately, alone or altogether, depending on the severity of the patient's illness.

In alternative embodiments, individuals or patients to whom a drug combination or composition or formulation as provided herein can be: (1) individuals or patients having been in contact with an infected person but are asymptomatic, or (2) individuals or patients that have been diagnosed by a blood test (i.e., are positive for virus) but are asymptomatic, or (3) individuals or patients that are symptomatic, for example, that have fever, sore throat, cough, chest pain, dyspnea and/or diarrhea, or are severely ill with high fever, aches and pains, unable to breathe to walk and/or barely maintaining consciousness.

In alternative embodiments, a drug combination or composition or formulation as provided herein is administered prophylactically, for example, to individuals who should or want to take the medication with them when travelling to prevent falling ill, for example, to prevent acquiring the infection when away from their doctor, country, or language.

In alternative embodiments, a drug combination or composition or formulation as provided herein is packaged and/or administered as a product of manufacture such as a blister pack or equivalent. In alternative embodiments, the blister pack or equivalent is designed to cover various stages of the infection, or to be for prophylactic purposes.

Alternatively the compounds may be solubilized, and then filtered with 22 micron filters or equivalents, suspended in a sterile fashion in saline or water or equivalents and administered intravenously, for example, in emergencies.

In alternative embodiments, methods comprise formulating and/or administering chloroquine (optionally, ARALEN™), chloroquine phosphate, chloroquine diphosphate, hydroxychloroquine (optionally, PLAQUENIL™) intravenously at about 300 mg, or between about 50 mg and 500 mg, in single dosages, or the equivalent thereof as an infusion.

In alternative embodiments, methods comprise formulating and/or administering chloroquine (optionally, ARALEN™), chloroquine phosphate, chloroquine diphosphate, hydroxychloroquine (optionally, PLAQUENIL™) at about 2.5 mg/kg intramuscularly (IM) at e.g., 0, 1, 12, 23, 24 and 25 hours, or e.g., at one or multiple dosages for between about one to two days or one day to two weeks.

In alternative embodiments, methods comprise formulating and/or administering chloroquine (optionally, ARALEN™), chloroquine phosphate, chloroquine diphosphate, hydroxychloroquine (optionally, PLAQUENIL™) at about 5 mg/kg subcutaneously (SC), for example, at 0, 12 and 24 hours, or for example, at one or multiple dosages for between about one to two days or one day to two weeks.

In alternative embodiments, oral administration of chloroquine (optionally, ARALEN™), chloroquine phosphate, chloroquine diphosphate, hydroxychloroquine (optionally, PLAQUENIL™) follows or complements (for example, is delivered together with) the IM or SC administration, or with the aerosol spray, mist, or powder administration of chloroquine (optionally, ARALEN™), chloroquine phosphate, chloroquine diphosphate, hydroxychloroquine (optionally, PLAQUENIL™), for example, as described below. In alternative embodiments, oral administration is dosaged at about 5 mg/kg, for example, for between about 12 and 72 hours (h), or for between about 36 and 48 h.

In alternative embodiments, methods comprise first administering (alone) for about 7 days (or 2, 3, 4, 5, 6 or 7 or more days) oseltamivir (or TAMIFLU™) at 3 times per day (tid) (or alternatively twice a day (bid) or four times a day or more), followed by a blood sample to be taken for virus testing; this initial administration dampens or slows a possible virus infection developing in the individual, so possibly ending up with a milder disease course, for example, where the side effects would be milder.

If and when virus blood positivity is confirmed, or viral infection is otherwise confirmed, the osteltamivir is supplemented by the lopinavir and ritonavir (which can be administered together or separately) three times daily (tid) (or alternatively twice a day (bid) or four times a day or more), while continuing the osteltamivir, for example, all three medications three (or two or four or more) times daily or 9 (or more) medications daily.

In alternative embodiments, the duration of the combined drug therapy as provided herein is 2, 3, 4, 5, 6, 7, 8, 9 or 10 or more days, which can be prolonged in those whose blood coronavirus remains positive longer with daily blood tests (or equivalent tests to confirm continued active infection), until the infection is shown to be gone or substantially diminished, particularly when the patient has symptomatically otherwise substantially recovered.

In alternative embodiments, other anti-coronavirus medications for example, listed above (optionally, molnupiravir, efavirenz (optionally, SUSTIVA™), nevirapine (or the combination efavirenz with emtricitabine and tenofovir, or ATRIPLA™), amprenavir (optionally, AGENERASE™), nelfinavir (optionally, VIRACEPT™) and/or remdesivir (optionally, GS-5734™, Gilead Sciences)) are added or mixed into the ‘cocktail’, for example, are mixed into osteltamivir, lopinavir and/or ritonavir formulations or one, several or all are administered separately.

In alternative embodiments, the contents of a blister pack or equivalent as provided herein have arranged thereof a combination of drugs (for example, as pill, capsules, tablets) to facilitate the patient's self-administration of a drug regimen as provided herein.

In alternative embodiments, an individual (for example, a patient) is given one, several or all of these medications (as provided in drug combinations or formulations as provided herein or used in methods as provided herein) in the form of a tablet, a capsule, a liquid, a spray, a powder, via an enema, as a suppository, administered subcutaneously or intravenously where available. When the patient is on a life support system the drug combination can be given parenterally.

Products of manufacture and Kits

Provided are products of manufacture and kits for practicing methods as provided herein; and optionally, products of manufacture and kits can further comprise instructions for practicing methods as provided herein.

Provided are compositions, including preparations, formulations and/or kits, comprising combinations of ingredients, for example, therapeutic combinations as described herein. In alternative embodiments, therapeutic combination can be mixed and administered together, or alternatively, they can be an individual member of a packaged combination of ingredients, for example, a liquid component and a solid product component manufactured in a separate compartment, package, kit or container; for example, where all or a subset of the combinations of ingredients are manufactured in a separate compartment, package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are for blister packs, clamshells or trays comprising a drug combination or formulation as provided herein, or a drug combination, pharmaceutical preparations or pharmaceutical compositions used to practice methods as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack comprises a moulded PVC base, with raised areas (the “blisters”) to contain the tablets, pills, etc. comprising the combinations of drugs drug combination, or formulations, pharmaceutical preparations or pharmaceutical compositions used in methods as provided herein, covered by a foil laminate. Tablets, pills, etc. can be removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one embodiment, provided is a method of packaging wherein the compositions comprising combinations of ingredients are contained in-between a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, for example, for items such as pills, tablets, geltabs, etc. are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two or three or more components: a thermoformed “blister” which houses multi-ingredient combination as provided herein, and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (for example, using an AERGO 8 DUO™, SCA Consumer Packaging, Inc., DeKalb Ill.) using regular heat seal tooling. This alternative aspect, using heat seal tooling, can seal common types of thermoformed packaging.

In alternative embodiments, therapeutic combinations and formulations drug combination, or pharmaceutical preparations or pharmaceutical compositions used in methods drug combination, are formulated, for example, as a powder, for example, as lyophilised material, for example, a lyophilized encapsulated product, for example, for practicing methods as provided herein, can be packaged alone or in combinations, for example, as “blister packages” or as a plurality of packettes, including as lidded blister packages, lidded blister or blister card or packets or packettes, or a shrink wrap.

In alternative embodiments, laminated aluminium foil blister packs are used, for example, for the preparation of therapeutic combinations or formulations as provided herein, or for pharmaceutical preparations or pharmaceutical compositions used in methods as provided herein. Products or kits comprise an aqueous solution(s) which are dispensed (for example, by measured dose) into containers. Trays can be freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/packettes are used, for example, using hard temper aluminium (for example, alufoil) lidding material. In one aspect, hermetically-sealed high barrier aluminium (for example, alufoil) laminates are used. In one aspect, products of manufacture include kits or blister packs, use foil laminations and strip packs, stick packs, sachets and pouches, peelable and non-peelable laminations combining foil, paper, or film for high barrier packaging.

In alternative embodiments, multi-component products of manufacture, including kits or blister packs as provided herein, include memory aids to help remind patients when and how to take the therapeutic combination. This safeguards the therapeutic combination's efficacy by protecting each tablet, geltab or pill until it's taken; gives the product or kit portability, makes it easy to take a dose anytime or anywhere.

Dosaging and Packaging for Therapeutic or Prophylactic Purposes

In alternative embodiments, provided are drug combinations and drug delivery devices comprising these combinations for therapeutic and/or prophylactic (prevention) purposes.

In alternative embodiments, a therapeutic or a prophylactic drug or ingredient combination “package”, which can be a blister pack, clamshell, or a nebulizer, inhaler, respirator or CPAP insert, or the like, is designed such that a particular drug or ingredient combination (for example, a drug or ingredient combination have 2, 3, 4, 5, or 6 ingredients or active agents, wherein one, several or all are separately formulated or formulated into one delivery agent such as a capsule or geltab, or nebulizer, inhaler, respirator or CPAP insert), to be taken by a user every day, every other day, every week, every two weeks or every 4 weeks (i.e., monthly). In alternative embodiments, the therapeutic or a prophylactic drug combination “package” is designed (for example, instructing the user) to take the drug combination as a staggered dosage, for example, one administration of the drug combination for two or three days in a row staggered by a week before the next two or three day administration cycle begins again.

In alternative embodiments, the therapeutic or prophylactic drug or ingredient combination comprises:

(1) (a) an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin, optionally at a dosage of between about 5, 6, 7, 8, 9 or 10 mg to 80 mg dosages, or 12 to 60 mg dosages; and (b) chloroquine (optionally, ARALEN™), chloroquine phosphate, chloroquine diphosphate or hydroxychloroquine (optionally, PLAQUENIL™);

(2) the combination of (1)(a) and (1)(b) also with vitamin D, vitamin D2 (or ergocalciferol), vitamin D3 (or cholecalciferol or calcifediol) optionally at a dosage of between about 3,000 to 100,000 units per day, or between about 10,000 to 50,000 units a day;

(3) the combination of (1)(a) and (1)(b) also with zinc (optionally a zinc sulphate, acetate, gluconate or picolinate) optionally at a dosage of between about 1 mg to 250 mg;

(4) the combination of (2)(a) and (2)(b) also with zinc (optionally a zinc sulphate, acetate, gluconate or picolinate) optionally at a dosage of between about 1 mg to 250 mg;

(5) the combination of (1)(a) and (1)(b) also with a tetracycline class drug, wherein optionally the tetracycline class drug comprises doxycycline, or DORYX™ DOXYHEXA™, DOXYLIN™, optionally dosages at between about 100 mg to 600 mg per day, optionally between about 200 mg to 400 mg;

(6) the combination of (2)(a) and (2)(b) also with a tetracycline class drug, wherein optionally the tetracycline class drug comprises doxycycline, or DORYX™ DOXYHEXA™, DOXYLIN™, optionally dosages at between about 25 mg to 600 mg per day, optionally between about 200 mg to 400 mg per day or between about 100 mg to 500 mg per day;

(7) the combination of (4) also with a tetracycline class drug, wherein optionally the tetracycline class drug comprises doxycycline, or DORYX™, DOXYHEXA™ DOXYLIN™, optionally dosages at between about 25 mg to about 600 mg per day, or between about 100 mg to 500 mg per day, optionally between about 200 mg to about 400 mg per day.

Inhalers, Nebulizers, Puffers and Nasal Sprays

In alternative embodiments, provided are drug delivery devices comprising an inhalation device or inhaler or aerosol or a nasal spray device, for example, a nebulizer, a puffer (as for asthma) or a modified hair dryer and the like, for the delivery of a therapeutic combination of drugs, a pharmaceutical dosage form or a formulation as provided herein. In alternative embodiments, provided are methods for administering a therapeutic combination of drugs, a pharmaceutical dosage form or a formulation as provided herein using an inhalation device, nebulizer, puffer or inhaler or a nasal spray device, for example, for the delivery of chloroquine (optionally, ARALEN™), chloroquine phosphate, chloroquine diphosphate, hydroxychloroquine (optionally, PLAQUENIL™), lopinavir, ritonavir and/or oseltamivir, and/or an anti-viral drug or medication or an anti-microbial drug as provided herein, or an anti-viral or an anti-microbial drug as used to practice methods as provided herein.

In alternative embodiments, the inhaler, nebulizer, puffer or the nasal spray device is a hand-held or otherwise portable (for example, worn around the neck) inhaler or a nasal spray device, and optionally the inhaler or a nasal spray device is a metered or dose-counting inhaler or a nasal spray device. In alternative embodiments, the inhaler or the nasal spray device is a device as described in for example, U.S. Pat. No. 10,583,261, or 10,561,809 (describing a breath actuated dry powder inhaler with a single air circulation chamber for de-agglomeration of entrained powdered medicament), or U.S. Pat. No. 10,561,807 (describing inhaler devices configured for consuming a defined capacity and generate an aerosol spray, mist, or aerosol imparted with flavor, a sensor configured to detect a predefined variable, an interface configured to make a notification to an inhaler of the aerosol, spray or mist, and a controller), or U.S. Pat. No. 10,463,815 (describing a dry powder inhaler may include a powder storage region, an inlet channel, a dispersion chamber, and an outlet channel); or U.S. patent application publication no. 20200069897 (describing inhalers having a breath actuated trigger mechanism reactive to an inhalation flow to trigger the release of a substance to be inhaled); or 20200061314 (describing a smart inhaler device having a flow pathway comprising a cartridge receptacle that is able to house a cartridge, flow meter, pump, and vaporizer; a wireless communication module; and at least one sensor that captures identifying information related to the cartridge); or 2020004691 (describing dry powder inhalers having replaceable cartridges containing a dry powder for local or systemic delivery through the pulmonary tract and lungs); or 20200046916 (describing an inhaler having a refill assembly comprising: a patient port; a canister actuable by the reusable assembly to deliver a dose of medicament to the patient port, a sleeve which is selectively actuable by a user independently of the reusable assembly so as to act on the canister to deliver a dose of medicament); or 20200046029 (describing an apparatus for generating an aerosol, spray or mist, and/or a vapour in an inhaler device includes a reservoir for storing a supply of a liquid; a heating system fluidly connected with the reservoir for receiving the liquid and configured to heat the liquid to generate the aerosol, spray or mist and/or vapor therefrom; a pumping system configured to pump the liquid from the reservoir to the heating system; and a valve arrangement for regulating flow from the pumping system to the heating system); or 20200016345 (describing a dry powder inhaler having a first chamber having an orifice for holding a dry powder and a gas, and a second chamber directly connected to the first chamber by at least one passageway for receiving an aerosolized form of the dry powder from in the first chamber and delivering the aerosolized dry powder to a user). An inhaler as provided herein, or as used in methods as provided herein, can comprise use of a dose counter, for example, as described in U.S. Pat. No. 10,561,808.

In alternative embodiments, the inhaler or the nasal spray device is a hand-held or otherwise portable inhaler or a nasal spray device is used or intended for use on public transport such as buses, trams, trains, aircraft and/or boats, or in places of commerce such as stores, bars, sporting events, movies theaters, theater, musical events, or any gathering of people.

In alternative embodiments, a drug or drug combination or a formulation as provided herein are delivered as a liquid, powder, spray or a mist through an oxygen tubing or an inhalation device such a CPAP (continuous positive air pressure) device, e.g., as used in sleep apnea treatment, a respirator or an ventilator.

In alternative embodiments, CPAP devices for delivering a drug or drug combination as provided herein can comprise components or be fabricated as, or be used, as described in e.g., U.S. Pat. Nos. 10,595,814; 10,549,057 (describing a ventilator system includes a mask to be placed over a wearer's face); 10,543,333 (describing a vent arrangement for a mask or associated conduit to discharge exhaled gas from the mask); and/or 10,406,312 (describing a CPAP flow driver for using nebulizer with CPAP apparatus).

In alternative embodiments, a medical device for inhalation delivery of a drug or a medication or combination as provided herein comprises a dry powder inhaler (such as a dry powder disk inhaler, e.g., as a DISKUS™ device), optionally having a dose counter window so user can see how many doses are left), e.g., where the powder is dose dispensed by (using) a disposable, refillable or replaceable cassette, packette or disk; and the dry powder dispensing can be breath activated, e.g., as an AEROLIZER™, FLEXHALER™, PRESSAIR™, DISKUS™, HANDIHALER™ TWISTHALER™, ELLIPTA™, NEOHALER™, RESPICLICK™, ROTAHALER™ or TUBUHALER™ device.

In alternative embodiments, the chloroquine (optionally, ARALEN™) chloroquine phosphate, chloroquine diphosphate, hydroxychloroquine (optionally, PLAQUENIL™), lopinavir, ritonavir and/or oseltamivir, and/or the anti-viral drug or drug combination or medication as provided herein, or anti-microbial drug as provided herein, is formulated as a powder (for example, a dry powder), a microparticle or a nanoparticle, or an aerosol, spray or mist. In alternative embodiments, the powder can be an agglomeration of powder particles or an agglomerate having irregular geometries such as width, diameter, and length. In alternative embodiments, the dry powder can be formulated as a granule of a physiologically acceptable excipient to be used as a carrier for a dry powder formulation for inhalation as described for example, in U.S. Pat. No. 10,583,085.

In alternative embodiments, the chloroquine (optionally, ARALEN™) chloroquine phosphate, chloroquine diphosphate, hydroxychloroquine (optionally, PLAQUENIL™) is formulated at between about 50% to 100% concentration as a liquid or aqueous formulation. In alternative embodiments, the chloroquine (optionally, ARALEN™), chloroquine phosphate, chloroquine diphosphate, hydroxychloroquine (optionally, PLAQUENIL™) is formulated and delivered at a dosage regimen of about 0.2 mg/kg to about 150 mg/kg per dose.

In alternative embodiments, methods of delivery of the chloroquine (optionally, ARALEN™), chloroquine phosphate, chloroquine diphosphate, hydroxychloroquine (optionally, PLAQUENIL™), lopinavir, ritonavir and/or oseltamivir, and/or the anti-viral drug or medication, or anti-microbial drug, comprise treatment regimens where the drug, medication or combination of drugs are administered every hour, every other hour, once, twice, three, four, five, six, seven, eight, nine, ten, eleven or twelve times a day. In alternative embodiments, the length of time of treatment, or the exact dosaging or dosage regimen, is determined by the clinician, or the administration is to begin immediately after possible exposure to an individual having (or exposed to another individual having) a coronavirus infection, or a COVID-19 or a 2019-nCoV (or so-called Wuhan coronavirus) infection, or an infection caused by a virus in the subfamily Orthocoronavirinae, or a virus in the family Coronaviridae, or a virus in the order Nidovirales.

In alternative embodiments, the chloroquine (optionally, ARALEN™) chloroquine phosphate, chloroquine diphosphate, hydroxychloroquine (optionally, PLAQUENIL™) is formulated at between about 50% to 100% concentration as a liquid or aqueous formulation, which can be used either as an aerosol, spray or mist and/or given orally. In alternative embodiments, the chloroquine (optionally, ARALEN™), chloroquine phosphate, chloroquine diphosphate, hydroxychloroquine (optionally, PLAQUENIL™) is formulated and delivered (for example, by inhalation and/or orally) at a dosage regimen of about 0.2 mg/kg to about 150 mg/kg per dose.

In alternative embodiments, the inhaled or aerosol formulation comprises an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin.

QT Interval Monitoring

In alternative embodiments of methods as provided herein, for patients being administered drugs or drug combinations as provided herein, or patients treated with methods as provided herein, the patient's QT interval is intermittently (for example, tid, bid or daily) or continuously monitored for any possible abnormality, particularly for QT interval prolongation, and if a QT abnormality, for example, QT interval prolongation, is found, then the amount (dosage) or frequency of the drug or drugs (for example, azithromycin) being administered is decreased, temporarily halted, or completed stopped. This alternative embodiment particularly applies to patient being administered chloroquine (optionally, ARALEN™), chloroquine phosphate, chloroquine diphosphate or hydroxychloroquine (optionally, PLAQUENIL™) and azithromycin (optionally, ZITHROMAX™, or AZITHROCIN™, optionally an oral extended-release formulation of azithromycin, or ZMAX™).

The QT interval is a measurement made on an electrocardiogram used to assess electrical properties of the heart, and the QT interval is calculated as the time from the start of a Q wave to the end of a T wave, which is approximately the time lapsed from when cardiac ventricles start to contract to when they finish relaxing. An abnormally long or abnormally short QT interval is associated with an increased risk of developing abnormal heart rhythms and sudden cardiac death.

For example, considering that a normal adult QT has baseline average of 435±24 milliseconds (ms) to a maximal average value of 463±32 ms, and that a QT measurement of greater than 500 ms is a known marker of malignant arrhythmia and sudden cardiac death, drug treatment (for example, azithromycin administration) in patients monitored to have a QT interval of about 500 or more ms or greater will be immediately ceased, and drug treatment in patients monitored to have a QT interval of about 450 ms to 490 ms will be modified by decreasing the dosage of administered drug or drugs (for example, azithromycin) (for example, by about 50%), or halting drug administration until QT intervals return to baseline, or normal.

In alternative embodiments, chloroquine (or ARALEN™), chloroquine phosphate, chloroquine diphosphate and/or hydroxychloroquine (optionally, PLAQUENIL™) is administered the entire length of the treatment but the azithromycin (optionally, ZITHROMAX™, or AZITHROCIN™, optionally an oral extended-release formulation of azithromycin, or ZMAX™) administration is halted or ceased after two, three, four, five or six days after treatment is commenced to prevent or ameliorate QT prolongation (which generally becomes detectable by day 4 of the treatment), and optionally the azithromycin administration is replaced by a tetracycline class drug, wherein optionally the tetracycline class drug comprises doxycycline, or DORYX™, DOXYHEXA™, DOXYLIN™ administration; or, the chloroquine (or ARALEN™), chloroquine phosphate, chloroquine diphosphate and/or hydroxychloroquine is administered alone. In alternative embodiments, when azithromycin is ceased after the second, third, fourth, fifth or sixth day, the remaining drug treatment (optionally, chloroquine (or ARALEN™), chloroquine phosphate, chloroquine diphosphate and/or hydroxychloroquine is administered alone or with another antiviral antibiotic, optionally, a tetracycline class drug, and optionally the tetracycline class drug comprises doxycycline) the treatment can last as long as about 2 to three weeks, or for between about 20 to 50 days or more (or as long as the patient tests positive for virus).

In alternative embodiments, the azithromycin is administered at about 500 mg on day one of treatment followed by dose reduction to between about 200 to 300 mg, or 250 mg, for about 2, 3, 4 or 5 more days, after which azithromycin administration is ceased. In alternative embodiments of methods as provided herein, patients being administered drugs or drug combinations as provided herein, or patients treated with methods as provided herein, will be continuously monitored with a device capable of remote signaling to a health care provider, for example, by computer, phone or watch, any abnormal heart rhythm, for example, an abnormal QT interval. In alternative embodiments, the patient is connected to a device that reads and records the electrical impulses of the heart and transmits this information automatically to the patient and/or health care provider, for example, by way of the internet and wireless technology. In alternative embodiments, the device is an electrocardiogram electrocardiography (ECG) patch monitor, a Holter monitor, an implantable loop recorder, or wrist band device, a smart phone or smart watch. In alternative embodiments, the device comprises an ECG sensor and an computer program (or application or “app”) that includes an algorithm to detect atrial fibrillation and transmit and alert for its occurrence.

In alternative embodiments, the ECG patch monitor can be a ZIO™ patch, and can be an adhesive, single-lead ECG monitor that is applied to the left pectoral region, and can comprise a System on a Chip (SoC) that converts analog ECG signals to digital format, an accelerometer to assist with artifact removal, a low-power Blue Tooth low energy processor that transmits the data, and a lithium polymer battery.

Formulations and pharmaceutical compositions

In alternative embodiments, the invention provides pharmaceutical formulations or compositions for use in in vivo, in vitro or ex vivo methods to treat, prevent, reverse and/or ameliorate a viral infection, for example, coronavirus (optionally COVID-19).

In alternative embodiments, the pharmaceutical compositions as provided herein or used to practice methods as provided herein can be administered parenterally, topically, orally or by local administration, such as by aerosol or transdermally. These pharmaceutical compositions can be formulated in any way and can be administered in a variety of unit dosage forms depending upon the condition or disease and the degree of illness, the general medical condition of each patient, the resulting preferred method of administration and the like. Details on techniques for formulation and administration are well described in the scientific and patent literature, see, for example, the latest edition of Remington's Pharmaceutical Sciences, Maack Publishing Co., Easton Pa. (“Remington's”). For example, in alternative embodiments, these compositions of the invention are formulated in a buffer, in a saline solution, in a powder, an emulsion, in a vesicle, in a liposome, in a nanoparticle, in a nanolipoparticle and the like. In alternative embodiments, the compositions can be formulated in any way and can be applied in a variety of concentrations and forms depending on the desired in vivo, in vitro or ex vivo conditions, a desired in vivo, in vitro or ex vivo method of administration and the like. Details on techniques for in vivo, in vitro or ex vivo formulations and administrations are well described in the scientific and patent literature. Formulations and/or carriers used to practice methods as provided herein can be in forms such as tablets, pills, powders, capsules, liquids, gels, syrups, slurries, suspensions, etc., suitable for in vivo, in vitro or ex vivo applications.

In alternative embodiment, compounds (for example, formulations) as provided herein or used to practice methods as provided herein can comprise a solution of compositions disposed in or dissolved in a pharmaceutically acceptable carrier, for example, acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations as provided herein or used to practice methods as provided herein can comprise auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agent in these formulations can vary widely, and can be selected primarily based on fluid volumes, viscosities and the like, in accordance with the particular mode of in vivo, in vitro or ex vivo administration selected and the desired results.

The compositions and formulations as provided herein or used to practice methods as provided herein can be delivered by the use of liposomes. By using liposomes, particularly where the liposome surface carries ligands specific for target cells (for example, an injured or diseased neuronal cell or CNS tissue), or are otherwise preferentially directed to a specific tissue or organ type, one can focus the delivery of the active agent into a target cells in an in vivo, in vitro or ex vivo application.

Nanoparticles, Nanolipoparticles and Liposomes

Also provided are nanoparticles, nanolipoparticles, vesicles and liposomal membranes comprising compounds or mixtures of compounds as provided herein or used to practice methods as provided herein. For example, in alternative embodiments, an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™) is formulated and/or administered in a liposome or nanoparticle formulation.

In alternative embodiments, provided are multilayered liposomes comprising compounds or mixtures of compounds used to practice methods as provided herein, for example, as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice methods as provided herein.

Liposomes can be made using any method, for example, as described in Park, et al., U.S. Pat. Pub. No. 20070042031, including method of producing a liposome by encapsulating an active agent, the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice methods as provided herein comprise a substituted ammonium and/or polyanions, for example, for targeting delivery of a compound, as described for example, in U.S. Pat. Pub. No. 20070110798.

The invention also provides nanoparticles comprising compounds used to practice methods as provided herein in the form of active agent-containing nanoparticles (for example, a secondary nanoparticle), as described, for example, in U.S. Pat. Pub. No. 20070077286. In one embodiment, provided are nanoparticles comprising a fat-soluble active agent of this invention or a fat-solubilized water-soluble active agent to act with a bivalent or trivalent metal salt.

In one embodiment, solid lipid suspensions can be used to formulate and to deliver compositions used to practice methods as provided herein to mammalian cells in vivo, in vitro or ex vivo, as described, for example, in U.S. Pat. Pub. No. 20050136121.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

### Example 1: Exemplary Treatment Regimens

This example demonstrates that methods and products of manufacture as provided are effective for treating, preventing, ameliorating, slowing the progress of, decreasing the severity of or preventing a coronavirus infection, or a COVID-19 (or so-called Wuhan coronavirus) infection, or an infection caused by a virus in the subfamily Orthocoronavirinae, or a virus in the family Coronaviridae, or a virus in the order Nidovirales.

A 42 year (y) old female patient is first treated with osteltamivir (or TAMIFLU™) from the time she was in contact with a patient from China later found to be positive for a coronavirus, in particular, the COVID-19 virus (or so-called Wuhan coronavirus). She is treated for 4 days but then develops fever, cough, aches and some dyspnea, with blood result coming back positive for COVID-19 virus.

Because the patient remains positive for 2019-nCoV virus, treatment with lopinavir and ritonavir, or KALETRA™, ALTERA™, ALUVIA™, KALMELTREX, LOPIMUNE™ or LOPINAVIR™, is now added to the osteltamivir treatment, and within 48 hours her blood test becomes negative for the coronavirus.

This patient was quite ill reaching the dyspnoea phase and difficulty walking, and but for this new drug combination treatment would have been expected to die. The combination of drugs as provided herein can rapidly clear the coronavirus from the patient's blood, thereby terminating or significantly ameliorating an otherwise life-threatening illness.

### Example 2: Exemplary Treatment Regimen

A 47-year-old male returning from a trip develops draggles and muscle pains and a temperature of 37.5 C. He is tested for coronavirus and is found positive for COVID-19 on a swab test. He is commenced on a combination of twice-daily chloroquine to 250 mg, lopinavir 200 mg bid, retinovir 50 mg bid. together with 75 mg bid oseltamivir (optionally, TAMIFLU™).

His condition is further resolved within three days and he is negative on the swab test on day six. Muscle pains also disappear and he feels well with and is able to be released to go home from hospital on day seven.

### Example 3: Exemplary Treatment Regimen

A group of elderly travelers board a ship on a cruise, whereupon numerous individuals on the ship contract coronavirus COVID-19 on testing. This group is administered as a prophylactic therapy/treatment inhalant agents comprising two inhaled or aerosol doses, or twice daily, of 125 mg of chloroquine. None contract COVID-19 coronavirus and test negative for the virus upon arrival home after the cruise.

### Example 4: Exemplary Treatment Regimen

A 65-year-old female patient develops a respiratory infection with shortness of breath, and is admitted to a hospital, and is tested positive for COVID-19 coronavirus. She is treated with intravenous (IV) remdesivir 10 mg per kilogram, chloroquine 250 mg twice daily, inhaled interferon, and KALETRA™ (a lopinavir/ritonavir combination) 50/200 mg.

Over the next week the patient's shortness of breath first worsens, but then improves, having fewer muscle aches and shortness of breath. By day 8 her viral detection is negative for COVID-19. The patient is discharged on day 12 fully cured of the coronavirus using this exemplary drug combination.

### Example 5: Exemplary Treatment Regimen

Two Italian patients (one female, one male 69 years old with previous pulmonary disease) traveling in India tested positive for coronavirus COVID-19; they were administered lopinavir 200 mg twice daily, ritonavir 50 mg twice daily, chloroquine 250 mg twice daily, 75 mg oseltamivir (TAMIFLU™) twice daily, and the female patient tested negative after 7 days of this combined drug therapy, and the male patient showed significant improvement, with COVID-19 viral load diminished. No significant side effects from the administered drug combination were seen.

### Example 6: Exemplary Treatment Regimen

A 32-year-old male patient acquires nose swab positive coronavirus COVID-19 infection, possibly at a party. Symptoms include loss of the sense of taste, muscle aches, fever of 38.9C, sore throat cough and difficulty with breathing. He is seen by a COVID-19 specialist, who commences patient on a combination of opaganib or YELIVA™ tid at 200 mg or 300 mg per dose; hydroxychloroquine 200 mg twice daily; lopinavir 200 mg three times per day; ritonavir 50 mg three times per day, and oseltamivir 75 mg tid, where one, several or all of these drugs are administered orally, IM, IV and/or by inhalation individually or in combination (for example, as a single formulation where applicable). The patient progressively loses his fever, regains the sense of taste, and after further five days the cough and sore throat improve. Shortness of breath progressively improves but this requires more than 20 days (d) of continued treatment, yet the swabs are negative for COVID-19 on consecutive days by day 8.

### Example 7: Exemplary Treatment Regimen

A 72-year-old gentleman with a chronic cough suddenly develops fever, worsening of cough and shortness of breath walking up hills. He goes to the emergency room but the antibiotic that is given does not stop the progression of the illness. He sees a specialist who deals with respiratory disease and is found to be positive for COVID-19. He is commenced on opaganib or YELIVA™ tid at 200 mg or 300 mg per dose; and hydroxychloroquine 200 mg tid. He continues on this therapy and notices improvement within two days; a swab is negative on day seven and he is completely asymptomatic by day 20.

### Example 8: Exemplary Treatment Regimen

A 27-year-old female patient has a nose swab positive coronavirus COVID-19 infection. Symptoms include myalgia, sore throat cough and difficulty with breathing. She also complains of marked fatigue. The doctor commences her on a combination of hydroxychloroquine 200 mg twice daily (bid), lopinavir 200 mg three times per day (tid), ritonavir 50 mg tid, and oseltamivir 75 mg tid. The patient loses her fever after four days although the cough and sore throat continues for another two days. Shortness of breath progressively improves and after 12 days of treatment the swabs become negative on consecutive days.

### Example 9: Exemplary Treatment Regimen

A 73-year-old male develops loss of sense of smell and taste, followed by muscle aches and pains fever of 38.5° C. cough and a sore throat. He then notices he was short of breath and saw his doctor. The doctor commences him on hydroxychloroquine 200 mg twice daily increasing after three days to 3 times daily, together with azithromycin 50 mg three times a day, and within three days the patient's fever improves, then disappears. The patient continues on this composition of the two drugs for a total of 12 days and subsequent swabs are negative for coronavirus COVID-19.

### Example 10: Exemplary Treatment Regimen

A 49-year-old patient with nasopharyngeal swab proving an infection with coronavirus COVID-19, is treated to reduce symptoms using a combination of opaganib or YELIVA™ tid at 200 mg or 300 mg per dose, hydroxychloroquine 200 mg tid, azithromycin 50 mg bid. On day 4 of the treatment nasal swabs show an absence of the coronavirus, and this is also shown daily until day 14 showing a cure is achieved. The patient's symptoms abate fairly rapidly and he eventually becomes completely normal and asymptomatic.

### Example 11: Exemplary Prophylactic Treatment Regimens

Individuals are given an initial loading dosage of chloroquine, chloroquine phosphate, chloroquine diphosphate and/or hydroxychloroquine, which is administered or started at a high dose (for example, the so-called “loading dose”) for example, an oral (for example, a single dose such as a single tablet, capsule or pill), IV or IM dosage of between about 250 mg, 300 mg, 350 mg, 300 mg or 0.5 gram (gm) (500 mg) and 1.5 gm, or between about 400 mg and 1 gm, optionally with follow up administrations every 4 to 10 days, or every 1, 2, 3, 4, 5, 6, 7, 8, 9, 10 days or up to 20 or more days, at a lower dosage of between about 50 gm to 200 mg, or about 100 mg, total daily dosage, optionally continuing for between about one week to one month,

and the chloroquine, chloroquine phosphate, chloroquine diphosphate and/or hydroxychloroquine is administered together with:

a macrolide drug, optionally azithromycin, and optionally the macrolide drug is started with a high dose (for example, a so-called “loading dose”), optionally an oral (for example, a single dose such as a single tablet, capsule or pill), IV or IM dosage of between about 400 mg to 0.5 gram (gm) (500 mg) and 1 gm, or at about 500 mg, optionally with follow up administrations every 4 to 10 days, or every 1, 2, 3, 4, 5, 6, 7, 8, 9, 10 days or up to 20 or more days, at a lower dosage of between about 100 gm to 300 mg, or about 250 mg, total daily dosage, optionally continuing for between about one week to one month, and/or

opaganib or YELIVA™, wherein optionally the opaganib is administered at a dosage of QD (once a day), bid (twice a day) or tid (three times a day) at a dosage of between about 100 to 600 mg per day or per dosage, or at about 100, 200, 300, 400, 500 or 600 mg per day or per dosage, and/or

lopinavir combined (formulated) with ritonavir, or KALETRA™, ALTERA™, ALUVIA™, KALMELTREX, LOPIIVIUNE™ or LOPINAVIR™.

### Example 12

A patient diagnosed with coronavirus, for example, having COVID-19, using products of manufacture, drugs or drug combinations and/or methods as provided herein is treated with:

(1) hydroxychloroquine (optionally, PLAQUENIL™) is administered at a 400 bid (twice a day) loading dose on day one, the at 200 mg bid for the next nine or ten days;(2) azithromycin is administered (optionally, ZITHROMAX™, or AZITHROCIN™) at a 500 mg bid loading dose on day one, then 500 mg in the morning (MANE) for days two, three and four, then azithromycin ceased and replaced by doxycycline (optionally, DORYX™, DOXYHEXA™, DOXYLIN™) 100 mg bid for the remainder of the treatment (ten or eleven days), or

azithromycin is first administered (optionally, ZITHROMAX™, or AZITHROCIN™) at a 500 mg bid loading dose on day one, then 500 mg in the morning (MANE) for days two, three and four, then azithromycin ceased, and doxycycline 100 mg bid (or between about 25 to 500 mg bid) (optionally, DORYX™ DOXYHEXA™, DOXYLIN™) every day for the full duration of the treatment (ten or eleven days, or more); and

(3) zinc sulfate is administered at a dosage of 100 mg MANE every day of the treatment, wherein optionally the treatment lasts between about 10 days and 3 weeks, or 11 days and 2 weeks, or for about 10, 11, 12, 13 or 14 days.

### Example 13

A patient diagnosed with coronavirus, for example, having COVID-19, using products of manufacture, drugs or drug combinations and/or methods as provided herein is treated with:

(1) oseltamivir (optionally, TAMIFLU™) is administered 75 mg three times a day (tid, or tds), or oseltamivir is dosaged tid for a daily total amount of between about 225 mg per day to about 450 mg per day;

(2) ritonavir is administered 50 mg bid and lopinavir 200 mg bid, or lopinavir and ritonavir administered bid as one tablet, optionally, in the form of a KALETRA™ tablet, or in the form of heat stable granules, optionally in the form of heat stable pediatric granules (dosage can be adjusted by using a heat stable pediatric granulated form of KALETRA™);

(3) bismuth subcitrate is administered 300 mg bid; and

(4) zinc sulfate is administered 100 mg MANE every day of the treatment, wherein optionally the treatment lasts between about 5 days and 3 weeks, or 6 days and 2 weeks, or for about 7, 8, 9, 10, 11, 12, 13 or 14 days.

### Example 14

A patient diagnosed with coronavirus, for example, having COVID-19, using products of manufacture, drugs or drug combinations and/or methods as provided herein is treated with:

(1) hydroxychloroquine (optionally, PLAQUENIL™) is first administered at a 400 bid (twice a day) loading dose on day one, then is administered at 200 mg bid for the remainder of the treatment, wherein optionally the treatment lasts between about 5 days and 3 weeks, or 6 days and 2 weeks, or for about 7, 8, 9, 10, 11, 12, 13 to 14 days,

(2) doxycycline (optionally, DORYX™, DOXYHEXA™, DOXYLIN™) is administered at between about 25 to about 600 mg bid, or between about 50 to about 500 mg bid, between about 100 to about 500 mg bid, or about 100 mg bid;

(3) ribavirin or tribavirin (or COPEGUS™, REBETOL™, or VIRAZOLE™) administered at 400 mg in the morning (MANE), and 600 mg at night (NOCTE);

(4) favipiravir (or T-705, avigan, or favilavir) 800 mg bid; and

(5) zinc sulfate 100 mg MANE, wherein optionally the treatment lasts between about 5 days and 3 weeks, or 6 days and 2 weeks, or for about 7, 8, 9, 10, 11, 12, 13 to 14 days,

### Example 15

In alternative embodiments, provided are formulations or methods of administration of drug regimens comprising co-formulation or co-administration of: hydroxychloroquine (optionally, PLAQUENIL™), an avermectin class drug such as ivermectin (optionally STROMECTOL™), moxidectin (optionally CYDECTIN™, EQUEST™, QUEST™), selamectin (optionally STRONGHOLD™), a milbemycin (optionally milbemectin, milbemycin oxime, moxidectin or nemadectin), doramectin (optionally DECTOMAX™), eprinomectin or abamectin; zinc (Zn); vitamin (Vit) D3; and, vitamin C, or any combination thereof, for example hydroxychloroquine, Vitamin C, Vitamin D (optionally cholecalciferol, vitamin D3 or calcifediol), and Zinc.

In alternative embodiments, provided are formulations or methods of administration of drug regimens comprising co-formulation or co-administration of hydroxychloroquine (optionally, PLAQUENIL™), azithromycin (optionally, ZITHROMAX™, or AZITHROCIN™, optionally an oral extended- or delayed-release formulation of azithromycin, or ZMAX™)), vitamin C, vitamin D (optionally cholecalciferol, vitamin D3 or calcifediol), and zinc, or any combination thereof.

In alternative embodiments, any or all of this therapeutic combination is administered orally and/or by inhalation (for example, by use of a nebulizer or equivalent), for example, ivermectin can be inhaled and the remainder of the drug combination is taken orally.

In alternative embodiments, provided are exemplary formulations or methods of administration of drug regimens as set forth below (Arm A and Arm B being separate exemplary treatment regimens), where the numbers are in milligrams (mgs), and each column represents a day (i.e., the first column is day 1, the last column is day 10):

| ARM A | | | | | | | | | | |
|---|---|---|---|---|---|---|---|---|---|---|
| Hydroxycloroquine | 800 | 400 | 400 | 400 | 400 | 400 | 400 | 400 | 400 | 400 |
| Ivermectin | 12 | | | | | | 12 | | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |
| ARM B | | | | | | | | | | |
| Doxycycline | 200 | 200 | 200 | 200 | 200 | 200 | 200 | 200 | 200 | 200 |
| Zn | 40 | 40 | 40 | 40 | 40 | 40 | 40 | 40 | 40 | 40 |
| Vit D3 | 5000 | 5000 | 5000 | 5000 | 5000 | 5000 | 5000 | 5000 | 5000 | 5000 |
| Vit C | 1000 | 1000 | 1000 | 1000 | 1000 | 1000 | 1000 | 1000 | 1000 | 1000 |
| Ivermectin | 12 | | | | | | 12 | | | |
| Bismuth subsalicylate | 1048 | 1048 | 1048 | 1048 | 1048 | 1048 | 1048 | 1048 | 1048 | 1048 |

A number of embodiments of the invention have been described. Nevertheless, it can be understood that various modifications may be made without departing from the spirit and scope of the invention. Accordingly, other embodiments are within the scope of the following claims.

1-16. (canceled)

17. A therapeutic combination of drugs comprising: ivermectin;
an antibiotic, and zinc.

18. The therapeutic combination of claim 17, wherein the antibiotic comprises a tetracycline class drug.

19. The therapeutic combination of claim 18, wherein the tetracycline class drug comprises doxycycline.

20. The therapeutic combination of claim 19, comprising between about 25 mg to about 600 mg doxycycline.

21. The therapeutic combination of claim 17, wherein the antibiotic comprises azithromycin.

22. The therapeutic combination of claim 21, comprising between about 50 mg to about 2000 mg azithromycin.

23. The therapeutic combination of claim 21, wherein the azithromycin comprise an oral extended-release formulation of azithromycin.

24. The therapeutic combination of claim 17, wherein the zinc comprises a zinc sulphate, a zinc acetate, a zinc gluconate or a zinc picolinate.

25. The therapeutic combination of claim 17, comprising between about 1 mg to about 250 mg zinc.

26. The therapeutic combination of claim 17, further comprising a vitamin.

27. The therapeutic combination of claim 26, wherein the vitamin comprises vitamin D or cholecalciferol.

28. The therapeutic combination of claim 27, comprising between about 3,000 to about 100,000 units vitamin D or cholecalciferol.

29. The therapeutic combination of claim 28, comprising between about 10,000 to about 50,000 units vitamin D or cholecalciferol.

30. The therapeutic combination of claim 26, wherein the vitamin comprises vitamin C.

31. The therapeutic combination of claim 17, formulated as a liquid or an aerosol.

32. The therapeutic combination of claim 17, formulated as a powder.

33. The therapeutic combination of claim 17, formulated as a tablet, capsule, tablet or geltab.

34. The therapeutic combination of claim 17, formulated as an injectable formulation, or an intramuscular (IM) or intravenous (IV) formulation.

35. The therapeutic combination of claim 17, further comprising hydrocortisone, cortisol or dexamethasome.

36. The therapeutic combination of claim 17, further comprising hydroxychloroquine.
